SEQUENCE LISTING (5 sequences)

SEQ ID NO: 1 - protein, 135 aa, hepatitis C virus
YEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNW

SEQ ID NO: 2 - protein, 278 aa, hepatitis C virus
GVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSIDSTSILGIGTVLDQAETAGARLVVLATATPPGSVTVPHPNIEEVALSSTGEIPFYGKAIPIEVIKGGRHLIFCHSKKKCDELAAKLSGFGINAVAYYRGLDVSVIPTSGDVVVVATDALMTGFTGDFDSVIDCNTCVTQTVDFS

SEQ ID NO: 3 - protein, 14 aa, hepatitis C virus
  MISC_FEATURE (7)..(7): Xaa may be Val or Ala
ATCXNGXCWTVYHG

SEQ ID NO: 4 - protein, 9 aa, hepatitis C virus
  MISC_FEATURE (2)..(2): Xaa may be Ile, Val or Thr
CXNGXCWTV

SEQ ID NO: 5 - protein, 300 aa, hepatitis C virus
  MISC_FEATURE (5)..(5): Xaa may be Ile, Val or Thr
MATCXNGXCWTVYHGRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSIDSTSILGIGTVLDQAETAGARLVVLATATPPGSVTVPHPNIEEVALSSTGEIPFYGKAIPIEVIKGGRHLIFCHSKKKCDELAAKLSGFGINAVAYYRGLDVSVIPTSGDVVVVATDALMTGFTGDFDSVIDCNTCVTQTVDFS

(19) United States
(12) Patent Application Publication
(10) Pub. No.: US 2004/0151735 A1
(43) Pub. Date: Aug. 5, 2004

(54) HCV compositions

(75) Inventors: Geert Maertens, Brugge (BE); Erik Depla, Destelbergen (BE); Erik D'hondt, Kruibeke (BE)

Correspondence Address: NIXON & VANDERHYE, PC, 1100 N GLEBE ROAD, 8TH FLOOR, ARLINGTON, VA, 22201-4714, US

(73) Assignee: INNOGENETICS, Ghent (BE)

(21) Appl. No.: 10/703,086

(22) Filed: Nov. 7, 2003

Related U.S. Application Data
(60) Provisional application No. 60/424,675, filed on Nov. 8, 2002.

(51) Int. Cl. A61K 39/29; C07K 14/02

(52) U.S. Cl. 424/189.1; 530/350

(57) ABSTRACT

The invention relates to immunogenic and vaccine compositions useful in prophylactic and therapeutic treatment of HCV infection. More specifically, said compositions comprise a HCV envelope peptide and a HCV non-structural peptide.

The present application claims benefit of U.S. Provisional Application No. 60/424,675, filed Nov. 8, 2002, the entire contenets of which is incorporated herein by reference.

### FIELD OF THE INVENTION

The invention relates to the field of immunogenic and vaccine compositions useful in prophylactic and therapeutic treatment of HCV infection. More specifically, said compositions comprise a HCV envelope peptide and a HCV non-structural peptide.

### BACKGROUND OF THE INVENTION

The ca. 9.6 kb single-stranded RNA genome of the HCV virus comprises a 5′- and 3′-non-coding region (NCRs) and, in between these NCRs a single long open reading frame of ca. 9 kb encoding a HCV polyprotein of ca. 3000 amino acids.

HCV polypeptides are produced by translation from the open reading frame followed by proteolytic processing of the resulting ca. 330 kDa polyprotein. Structural proteins are derived from the amino-terminal one-fourth of the polyprotein and include the capsid or Core protein (ca. 21 kDa), the E1 envelope glycoprotein (ca. 31 kDa) and the E2 envelope glycoprotein (ca. 70 kDa), previously called NS1. From the remainder of the HCV polyprotein the non-structural HCV proteins are derived which include NS2 (ca. 23 kDa), NS3 (ca. 70 kDa), NS4A (ca. 8 kDa), NS4B (ca. 27 kDa), NS5A (ca. 58 kDa) and NS5B (ca. 68 kDa) (Grakoui et al. 1993). The E2 protein can occur with or without a C-terminal fusion of the p7 protein (Shimotohno et al. 1995). Recently, an alternative open reading frame in the Core-region was found which is encoding and expressing a ca. 17 kDa protein called F (Frameshift) protein (Xu et al. 2001; Ou & Xu in U.S. Patent Application Publication No. US2002/0076415). In the same region, ORFs for other 14-17 kDa ARFPs (Alternative Reading Frame Proteins), A1 to A4, were discovered and antibodies to at least A1, A2 and A3 were detected in sera of chronically infected patients (Walewski et al. 2001).

HCV is the major cause of non-A, non-B hepatitis worldwide. Acute infection with HCV (20% of all acute hepatitis infections) frequently leads to chronic hepatitis (70% of all chronic hepatitis cases) and end-stage cirrhosis. It is estimated that up to 20% of HCV chronic carriers may develop cirrhosis over a time period of about 20 years and that of those with cirrhosis between 1 to 4%/year is at risk to develop liver carcinoma. (Lauer & Walker 2001, Shiffman 1999). An option to increase the life-span of HCV-caused end-stage liver disease is liver transplantation (30% of all liver transplantations world-wide are due to HCV-infection).

Only limited information is available on the pathological mechanisms of liver damage and of virus clearance. As a first step towards the development of an effective prophylactic and/or therapeutic vaccine against HCV much efforts have been invested in identifying HCV components involved in protective immunity (B-cell or humoral responses and T-cell or cellular responses).

Determining for whether clearance of the virus and resolution of disease or virus persistence and chronic disease are occurring, is thought to be the immune responses during the acute phase of HCV infection. Several studies seem to suggest that the vigor, breadth and maintenance of the immune responses, and possibly especially the T-cell/CTL response, early during infection may be of prime importance in order to resolve infection. (Diepolder et al. 1995, 1997; Missale et al. 1996; Cooper et al. 1999, Erickson et al. 2001). Spontaneous resolution of infection is, however, only occurring in approximately 30% of HCV-infected persons despite the detectable presence of antibodies to HCV proteins and HCV-specific T-cells (and thus despite a detectable immune response) in patients evolving to chronic infection.

The options for treating HCV infection are currently very limited and normally comprise a treatment regimen of the antiviral ribavirin and interferon-α (or pegylated interferon-α). The most optimal treatment regimen today (combination of pegylated interferon-α with ribavirin and with extension of the therapy based on genotype and viral load) results in severe side effects (about 25% of patients stop therapy prematurely), and of those able to complete the treatment schedule only 50% show a sustained response if they are infected with genotype 1, the most predominant genotype world-wide (Manns et al. 2001). In addition, this therapy is not advised for patients with pre-existing markers of anaemia, auto-immune diseases or a history of depression which are frequent conditions in HCV. Because of these and other medical complications up to 75% of the HCV patients are excluded from therapy today (Falck-Ytter et al. 2002). Schering-Plough calculated the number of people who have not responded to the current therapies to increase to 1 million by 2010 (J. Albrecht, Schering-Plough satellite symposium, EASL, Madrid, April 2002).

The options for preventing HCV infection are currently limited to screening of blood donations for the presence of HCV antibodies and/or viral RNA. An important number of new HCV infections occur, however, via unknown routes, via intravenous drug users, or via persons not aware of being carrier of the HCV virus. There thus is a clear and urgent need for agents useful in both prevention and treatment of HCV infection.

A HCV vaccine may be a DNA-based vaccine, a protein- or peptide-based vaccine, or a combination of a DNA-prime protein-boost vaccination may be applied.

DNA-prime protein-boost vaccination studies have been performed in mice for Core (Hu et al. 1999) and E2 (Song et al. 2000). Core DNA vaccination produced a predominantly IgM antibody production whereas protein boosting caused an increase in IgG antibody levels. The T-cell proliferation response induced by Core DNA-vaccination was increased by the protein boost. A CTL response was not observed when the Core protein alone was injected but was detectable both in DNA-primed and in DNA-primed protein-boosted vaccinations. For E2, both the antibody response and the CTL response elicited upon DNA vaccination were augmented by protein boosting.

Studies with protein-based HCV vaccines are very limited and include immunization of mice with fragments of Core (Shirai et al. 1996, Hu et al. 1999), E1 (Lopez-Diaz de Cerio et al. 1999), E2 (Nakano et al. in U.S. Patent Publication No. 2002/0119495; Houghton et al. in U.S. Patent Application Publication No. 2002/0002272), E1/E2 or E1/E2+Core (Drane et al. in International Patent Publication No. WO01/37869) and NS5 (Shirai et al. 1996).

Mice were primed either with NS5, NS5 covalently attached to a helper peptide (fragment of HIV gp160 protein) or Core and the CTL-response was subsequently measured after restimulation in the presence of NS5 or Core. A CTL-reponse was observed for the NS5-HIV fusion protein and the Core protein but not for the NS5 protein alone. The CTL-reponse to the NS5-HIV fusion protein was dependent on the adjuvant used: a saponin adjuvant (QS21) supported the CTL-response whereas complete Freund's Adjuvant did not. No proliferative response to NS5 was detected (Shirai et al. 1996). No CTL-response to Core was detected under the conditions as outlined by Hu et al. (1999).

Immunization of mice with an E1-peptide (amino acids 121-135) induced CD4⁺ Th1 cells as well as a long-lasting CD8⁺ CTL response which was also obtained in the absence of an adjuvant (Lopez-Diaz de Cerio et al. 1999).

A T-cell proliferation response was noted when mice were previously immunized with an E2 peptide lacking the N- and C-terminal parts and produced by insect cells. This response was only detectable when the E2 peptide was adjuvanted with QS21 or MPL-TDM but not when adjuvanted with alum. A humoral anti-E2 response was only detected when mice were immunized with E2 adjuvanted with QS21 (Nakano et al. in U.S. Patent Publication No. 2002/0119495). Mice injected with an E1/E2 heterodimeric complex did not mount a significant anti-E2 antibody response. When the E1/E2 was adjuvanted with MF59 or when Core-ISCOM was added, a detectable and comparable anti-E2 antibody response was observed. The humoral response to E2 was higher when mice were immunized with E2 peptide (adjuvanted with MF59) compared to when immunized with E2-expressing plasmid (Houghton et al. in U.S. Patent Application Publication No. 2002/0002272).

All of the above exploratory vaccinations were performed on rodents which are not animal model systems for HCV infection. The obtained results can therefore not a priori be extrapolated to primates such as macaques or to chimpanzees or humans of which the latter two are susceptible to HCV infection. Therefore, of more interest are prophylactic and therapeutic vaccinations performed on chimpanzees or therapeutic vaccinations of HCV-infected humans. E2 DNA-vaccinations of mice, macaques and chimpanzees were described in two studies of Forms et al. (1999, 2000). The humoral immune response (both in mice and macaques) occurred earlier and was stronger with an E2 variant expressed on the cell surface compared with an E2 variant expressed in the cytosol. Furthermore, the humoral response in macaques was lower than the response in mice despite injection of the macaques with 1 mg of plasmid DNA (Forms et al. 1999). In a follow-up study the cell surface-targeted E2 DNA-vaccine was administered to chimpanzees (3 times 10 mg of plasmid DNA). This vaccination did, upon challenge infection with 100 CID₅₀ (50% chimpanzee infectious doses) homologous monoclonal HCV, not result in sterilizing immunity although recovery from acute HCV infection was apparent. Interestingly, recovery from acute HCV infection was faster in the chimpanzee most likely infected with HCV before (Forns et al. 2000).

Rhesus macaques were injected with Core-expressing vaccinia virus, Core adjuvanted with LTK63 or Core adjuvanted with ISCOM in a study by Drane et al. (in International Patent Publication No. WO01/37869). A CD8⁺ CTL-response was elicited by immunization with Core-expressing vaccinia virus or with Core adjuvanted with ISCOM. Further analysis of the animals immunized with Core-ISCOM revealed a sustained CTL-response, a CD4⁺ T-cell proliferation response as well as a humoral response to Core.

Prophylactic vaccination of chimpanzees with an E1/E2 or Core/E1/E2 complex has been described in Choo et al. (1994), Houghton et al. (1995). Upon challenge infection with 10 CID₅₀ homologous HCV (HCV-1, of type 1a), the chimpanzees with the highest anti-E1/E2-antibody response to the vaccine at the time of challenge resolved the infection. No such correlation was apparent between levels of anti-E2 HVRI antibody levels and the outcome of viral challenge, this despite the reported existence of neutralization of binding antibodies against E2-HVRI (Ishii et al. 1998, Shimizu et al. 1994). Upon re-challenge, and after re-immunization of the vaccinated chimpanzees which resolved the first challenge infection with 64 CID₅₀ of a heterologous HCV (HCV-H, another type 1a-isolate), the chimpanzees became infected, although viremia was delayed.

Prophylactic and therapeutic vaccination of chimpanzees with an E1 protein has been described in WO99/67285 and WO02/055548. A therapeutic effect (decrease of ALT-levels, detectable E2-antigen and liver inflammation) was observed both in a heterologous setting within the same subtype (vaccine of type 1b E1 effective in chimpanzees infected with another type 1b HCV isolate) and in a cross-subtype setting (vaccine of type 1b E1 effective in chimpanzee infected with a type 1a HCV). The same vaccine also resulted in resolving of acute HCV-infection in vaccinated naïve chimpanzees challenged with 100 CID₅₀ of a heterologous type 1b HCV. Interestingly, the immune responses observed in chimpanzees were also observed in HCV-infected humans and in healthy volunteers.

From the above, it can be concluded that immune responses elicited by HCV proteins depend on various factors such as type of adjuvant and presence or absence of a helper peptide. The immune responses also differ between eliciatation by a combination of peptides versus elicitation by the peptides alone. In non-human primates, exploratory vaccinations have so far been performed only with Core (immune responses depending on adjuvant; prophylactic and therapeutic effects currently unknown), with an E1/E2 or Core/E1/E2 protein complex (prophylactic protection against homologous HCV), or with E1 (prophylactic and therapeutic effects). Although these exploratory vaccination studies are encouraging, vaccine compositions based on HCV protein combinations may result in a broader immune response and, thus, in improved prophylactic and/or therapeutic effects on HCV infection.

### SUMMARY OF THE INVENTION

In one aspect, the current invention relates to an HCV immunogenic composition comprising at least one HCV envelope peptide, at least one HCV non-structural peptide, and, optionally, a pharmaceutically acceptable carrier. Said HCV immunogenic composition may be a HCV vaccine composition comprising an effective amount of at least one HCV envelope peptide, at least one HCV non-structural peptide, and, optionally, a pharmaceutically acceptable carrier. Said HCV vaccine composition may be a prophylactic HCV vaccine composition and/or a therapeutic HCV vaccine composition comprising a prophylactically and/or therapeutically effective amount, respectively, of at least one HCV envelope peptide, at least one HCV non-structural peptide, and, optionally, a pharmaceutically acceptable carrier.

In particular the HCV immunogenic composition, HCV vaccine composition, prophylactic HCV vaccine composition and/or therapeutic HCV vaccine composition of the invention comprise a HCV E1 envelope peptide and a HCV NS3 non-structural peptide.

In one embodiment, the HCV immunogenic composition, HCV vaccine composition, prophylactic HCV vaccine composition and/or therapeutic HCV vaccine composition of the invention comprise a HCV E1 peptide that is consisting of the HCV polyprotein region spanning amino acids 192 to 326, and an HCV NS3 peptide that is comprising the HCV polyprotein region spanning amino acids 1188 to 1468. More particularly, said HCV NS3 peptide may further comprise the HCV polyprotein region spanning amino acids 1071 to 1084 or parts thereof, such as the HCV polyprotein region spanning amino acids 1073 to 1081. Said HCV NS3 peptide may also comprise the HCV polyprotein region spanning amino acids 1188 to 1468 and the HCV polyprotein region spanning amino acids 1071 to 1084 or parts thereof. In a further embodiment, the HCV immunogenic composition, HCV vaccine composition, prophylactic HCV vaccine composition and/or therapeutic HCV vaccine composition of the invention comprises an HCV E1 peptide defined by SEQ ID NO:1 and an HCV NS3 peptide comprising the HCV polyprotein region spanning amino acids 1188 to 1468 defined by SEQ ID NO:2. More particularly, said HCV NS3 peptide may further comprise the HCV polyprotein region spanning amino acids 1071 to 1084 defined by SEQ ID NO:3 or parts thereof, such as the HCV polyprotein region spanning amino acids 1073 to 1081, defined by, e.g., SEQ ID NO:4. Said HCV NS3 peptide may also comprise the HCV NS3 peptides defined by SEQ ID NO:2 and by SEQ ID NO:3 or SEQ ID NO:4. In particular, such HCV NS3 peptide may be defined by SEQ ID NO:5.

In a further aspect, the current invention relates to an HCV immunogenic composition, an HCV vaccine composition, a prophylactic HCV vaccine composition and/or a therapeutic HCV vaccine composition wherein any of said compositions is comprising, besides the optional pharmaceutically acceptable carrier, at least one HCV envelope peptide and at least one HCV non-structural peptide, and wherein said HCV peptides are linked, optionally via a spacer.

In a further aspect, the current invention relates to an HCV immunogenic composition, an HCV vaccine composition, a prophylactic HCV vaccine composition and/or a therapeutic HCV vaccine composition wherein any of said compositions is comprising, besides the optional pharmaceutically acceptable carrier, at least one HCV envelope peptide and at least one HCV non-structural peptide, and:

wherein said HCV peptides are synthetic peptides or recombinant peptides; and/or

wherein at least one cysteine of said HCV peptides is reversibly or irreversibly blocked; and/or

wherein at least one cysteine of said HCV envelope peptide is alkylated; and/or

wherein at least one cysteine of said HCV non-structural peptide is sulphonated; and/or

wherein said HCV envelope peptide is added to said composition as viral-like particles.

In another aspect, the current invention relates to an HCV immunogenic composition, an HCV vaccine composition, a prophylactic HCV vaccine composition and/or a therapeutic HCV vaccine composition wherein any of said compositions is comprising, besides the optional pharmaceutically acceptable carrier:

a plurality of HCV envelope peptides derived from different HCV genotypes, subtypes or isolates and at least one HCV non-structural peptide; or

at least one HCV envelope peptide and a plurality of HCV non-structural peptides derived from different HCV genotypes, subtypes or isolates; or

a plurality of HCV envelope peptides derived from different HCV genotypes, subtypes or isolates and a plurality of HCV non-structural peptides derived from different HCV genotypes, subtypes or isolates.

Further aspects of the current invention comprise the use of an HCV immunogenic composition, an HCV vaccine composition, a prophylactic HCV vaccine composition and/or a therapeutic HCV vaccine composition according to the invention for:

inducing in a mammal a humoral response to the HCV peptides comprised in any of said composition; and/or

inducing in a mammal a cellular response to the HCV peptides comprised in any of said composition, wherein said cellular response may be a CD4⁺ T-cell proliferation response and/or a CD8⁺ cytotoxic T-cell response and/or the increased production of cytokines; and/or

for prophylactic protection of a mammal against chronic HCV infection, wherein said HCV may be a homologous or a heterologous HCV; and/or

for therapeutically treating a chronically HCV-infected mammal, wherein said HCV may be a homologous or a heterologous HCV; and/or

for reducing liver disease in a HCV-infected mammal; and/or

for reducing liver disease in a chronic HCV-infected mammal by at least 2 points according to the overall Ishak score; and/or

for reducing serum liver enzyme activity levels in a HCV-infected mammal, wherein said liver enzyme may be, e.g., alanine aminotransferase (ALT) or gamma-glutamylpeptidase; and/or

for reducing HCV RNA levels in a HCV-infected mammal; and/or

for reducing liver fibrosis progression in a HCV-infected mammal; and/or

for reducing liver fibrosis in a HCV-infected mammal.

Said mammal obviously may be a human.

In particular, the uses according to the invention are methods for obtaining at least one of the recited effects, with said methods comprising administering any of said compositions to a mammal or a human.

Other aspects of the invention relate to methods of vaccinating a HCV-naïve or HCV-infected mammal comprising administering a DNA vaccine and an HCV immunogenic composition, an HCV vaccine composition, a prophylactic HCV vaccine composition and/or a therapeutic HCV vaccine composition according to the invention.

### FIGURE LEGENDS

FIG. 1. Schematic map of the vector pFPMT-CL-H6-K-E1s.

FIG. 2. Western blot analysis of HCV E1s protein produced by Hansenula E1s (lane 1) and by Vero cells (lane 2). Size of molecular weight markers (lane 3) are indicated on the right (kDa). The E1-specific murine monoclonal antibody IGH 201 (see International Patent Application Publication No. WO99/50301) was used for detection of the E1 proteins.

FIG. 3. E1 s-specific T cell stimulation observed at week 0 (black bars) or week 11 (hatched bars). T cell stimulation is expressed as stimulation index (SI) on the Y-axis for the rhesus monkeys indicated on the X-axis. Animals 1 to 4 were vaccinated with NS3 and Vero E1s and isolated PBMC restimulated in vitro with Vero E1s. Animals 5 to 8 were vaccinated with NS3 and yeast E1s and isolated PBMC restimulated in vitro with yeast E1s.

FIG. 4. NS3-specific T cell stimulation observed at week 0 (black bars) or week 11 (hatched bars). T cell stimulation is expressed as stimulation index (SI) on the Y-axis for the rhesus monkeys indicated on the X-axis. Animals 1 to 8 were vaccinated with NS3. Animals 1 to 4 were also vaccinated with Vero E1s. Animals 5 to 8 were also vaccinated with yeast E1s.

FIG. 5. E1s-specific T cell stimulation expressed as stimulation index (SI) on the Y-axis for the rhesus monkeys indicated on the X-axis. Animals 1 to 4 were vaccinated with NS3 and Vero E1s and isolated PBMC restimulated in vitro with Vero E1s (hatched bars) or yeast E1s (black bars). Animals 5 to 8 were vaccinated with NS3 and yeast E1s and isolated PBMC restimulated in vitro with Vero E1s (hatched bars) or yeast E1s (black bars).

### DETAILED DESCRIPTION OF THE INVENTION

Work leading to the present invention resulted in the unexpected effect that co-injection of an HCV envelope protein, more particularly E1, and an HCV non-structural protein, more particularly NS3, both in a formulation with the same adjuvant, elicited a strong humoral response and a strong cellular response to both of the HCV antigens in non-human primates. This immune response is broader than the immune responses obtained so far with exploratory HCV protein-based immunogenic/vaccine composition. The observed broad immune response therefore opens the way to formulate a HCV envelope protein antigen and a HCV non-structural protein antigen in a single immunogenic composition which can be used in mammals as vaccine composition, e.g. for therapeutic or prophylactic purposes.

Thus, in one aspect, the current invention relates to an HCV immunogenic composition comprising at least one HCV envelope peptide, at least one HCV non-structural peptide, and, optionally, a pharmaceutically acceptable carrier. Said HCV immunogenic composition may be a HCV vaccine composition comprising an effective amount of at least one HCV envelope peptide, at least one HCV non-structural peptide, and, optionally, a pharmaceutically acceptable carrier. Said HCV vaccine composition may be a prophylactic HCV vaccine composition and/or a therapeutic HCV vaccine composition comprising a prophylactically and/or therapeutically effective amount, respectively, of at least one HCV envelope peptide, at least one HCV non-structural peptide, and, optionally, a pharmaceutically acceptable carrier.

The term “immunogenic” refers to the ability of a protein or a substance to produce at least one element of an immune response. The immune response is the total response of the body of an animal to the introduction of an antigen and comprises multiple elements including antibody formation (humoral response or humoral immunity), cellular immunity, hypersensitivity, or immunological tolerance. Cellular immunity refers to cellular responses elicited by an antigen and include a T-helper cell- and/or CTL-response. The term “antigen” refers to the ability of a peptide, protein or other substance to be antigenic or immunogenic. An antigen is understood to comprise at least one epitope.

“Antigenic” refers to the capability of a protein or substance to be recognized by an elicited humoral and/or cellular immune response. Typically, the antigenic quality of a protein or substance is determined by in vitro assays. For humoral responses, a protein or substance can be referred to as antigenic in case the protein or substance is recognized by elicited antibodies in e.g. an ELISA, western-blot, RIA, immunoprecipitation assay or any similar assay in which the protein or substance is allowed to be recognized by an elicited antibody and in which such a recognition can be measured by, e.g., a colorometric, fluorometric or radioactive detection, or formation of a precipitate. For cellular response, a protein or substance can be referred to as antigenic in case the protein or substance is recognized by an elicited T-cell response in e.g. an T-cell proliferation assay, a ⁵¹Cr-release assay, a cytokine secretion assay or alike in which the protein or substance is incubated in the presence of T-cells drawn from an individual in which immune response have been elicited and in which a recognition by the T-cell is measured by, e.g., a proliferative repsonse, a cell lysis response, a cytokine secretion. An antigenic protein or substance may be immunogenic in se but may also require additional structures to be rendered immunogenic.

An “immunogenic composition” is a composition referred to as being immunogenic, i.e. a composition comprising an antigen capable of eliciting at least one element of the immune response against the antigen comprised in said composition when said composition is introduced into the body of an animal capable of raising an immune response. An immunogenic composition may clearly comprise more than one antigen, i.e., a plurality of antigens, e.g. 2, 3, 4, 5, 6, 7, 8, 9, 10 or more, e.g., up to 15, 20, 25, 30, 40 or 50 or more distinct antigens. In particular, the immunogenic composition of the invention is an HCV immunogenic composition wherein the antigens are HCV antigens such as HCV envelope protein antigens and/or HCV non-structural protein antigens.

A “vaccine composition” is an immunogenic composition capable of eliciting an immune response sufficiently broad and vigorous to provoke one or both of:

a stabilizing effect on the multiplication of a pathogen already present in a host and against which the vaccine composition is targeted; and

an effect increasing the rate at which a pathogen newly introduced in a host, after immunization with a vaccine composition targeted against said pathogen, is resolved from said host.

A vaccine composition may clearly also provoke an immune response broad and strong enough to exert a negative effect on the survival of a pathogen already present in a host or broad and strong enough to prevent an immunized host from developing disease symptoms caused by a newly introduced pathogen. In particular the vaccine composition of the invention is a HCV vaccine composition wherein the pathogen is HCV.

An “effective amount” of an antigen in a vaccine composition is referred to as an amount of antigen required and sufficient to elicit an immune response. It will be clear to the skilled artisan that the immune response sufficiently broad and vigorous to provoke the effects envisaged by the vaccine composition may require successive (in time) immunizations with the vaccine composition as part of a vaccination scheme or vaccination schedule. The “effective amount” may vary depending on the health and physical condition of the individual to be treated, the taxonomic group of the individual to be treated (e.g. human, non-human primate, primate, etc.), the capacity of the individual's immune system to mount an effective immune response, the degree of protection desired, the formulation of the vaccine, the treating doctor's assessment, the strain of the infecting pathogen and other relevant factors. It is expected that the amount will fall in a relatively broad range that can be determined through routine trials. Usually, the amount will vary from 0.01 to 1000 μg/dose, more particularly from 0.1 to 100 μg/dose. Dosage treatment may be a single dose schedule or a multiple dose schedule. The vaccine may be administered in conjunction with other immunoregulatory agents.

A “prophylactic vaccine composition” is a vaccine composition providing protective immunity, i.e., an immunity preventing development of disease upon challenge of the host immunized with the prophylactic vaccine composition. In particular for HCV, a prophylactic HCV vaccine composition is to be understood as a vaccine composition capable of providing protective immunity helping to resolve a challenge HCV infection rapidly and/or preventing a challenge HCV infection to proceed to a chronic infection. Accelerated HCV viral clearance or accelerated control of HCV challenge infection is thus envisaged by vaccination with a prophylactic HCV composition according to the invention.

A “prophylactically effective amount” of an antigen in a prophylactic vaccine composition is referred to as an amount of antigen required and sufficient to elicit an immune response enabling the development of protective immunity. It will be clear to the skilled artisan that the immune response sufficiently broad and vigorous to provoke the effects envisaged by the prophylactic vaccine composition may need require successive (in time) immunizations with the prophylactic vaccine composition (see also “effective amount”).

A “therapeutic vaccine composition” is a vaccine composition providing a curative immune response, i.e., an immune response capable of effectuating a reversion, or at least capable of effectuating halting, of disease symptoms associated with an already established pathogen infection. In particular for HCV, a therapeutic HCV vaccine composition is to be understood as a vaccine compositions capable of reducing serum liver enzyme, e.g., alanine aminotransferase (ALT) or γ-glutamylpeptidase (γ-GT), activity levels in the blood and/or of reducing HCV RNA levels and/or of reducing liver disease and/or of reducing liver fibrosis and/or of reducing liver fibrosis progression.

A “therapeutically effective amount” of an antigen in a therapeutic vaccine composition is referred to as an amount of antigen required and sufficient to elicit an immune response enabling the development of a curative immune response. It will be clear to the skilled artisan that the antigenic or immunogenic response sufficiently broad and vigorous to provoke the effects envisaged by the therapeutic vaccine composition may need require successive (in time) immunizations with the therapeutic vaccine composition (see also “effective amount”).

In particular the HCV immunogenic composition, HCV vaccine composition, prophylactic HCV vaccine composition and/or therapeutic HCV vaccine composition of the invention comprise a HCV E1 envelope peptide and a HCV NS3 non-structural peptide. Other combinations of HCV envelope peptides and HCV non-structural peptides are not excluded and comprise, e.g., E1 and NS2, E1 and NS4, E1 and NS4A, E1 and NS4B, E1 and NS5, E1 and NS5A, E1 and NS5B, E2 and NS2, E2 and NS4, E2 and NS4A, E2 and NS4B, E2 and NS5, E2 and NS5A, and E2 and NS5B.

With “HCV envelope peptide” is meant herein any HCV E1 or E2 protein, any fragment thereof, or any derivative thereof, which when comprised in an immunogenic composition, a vaccine composition, a therapeutic vaccine composition or a prophylactic vaccine composition, is capable of eliciting an immune response as defined for an immunogenic composition, a vaccine composition, a therapeutic vaccine composition or a prophylactic vaccine composition, respectively. More particularly, the immunogenic composition, vaccine composition, therapeutic vaccine composition or prophylactic vaccine composition is an HCV immunogenic composition, a HCV vaccine composition, a therapeutic HCV vaccine composition or a prophylactic HCV vaccine composition, respectively, according to the present invention.

With “HCV non-structural peptide” is meant herein any HCV NS2, NS3, NS4 or NS5 protein, any fragment thereof (e.g., NS4A, NS4B, NS5A, NS5B), or any derivative thereof, which when comprised in an immunogenic composition, a vaccine composition, a therapeutic vaccine composition or a prophylactic vaccine composition, is capable of eliciting an immune response as defined for an immunogenic composition, a vaccine composition, a therapeutic vaccine composition or a prophylactic vaccine composition, respectively. More particularly, the immunogenic composition, vaccine composition, therapeutic vaccine composition or prophylactic vaccine composition is an HCV immunogenic composition, a HCV vaccine composition, a therapeutic HCV vaccine composition or a prophylactic HCV vaccine composition, respectively, according to the present invention.

A derivative of a HCV peptide is meant to include HCV peptides comprising modified amino acids (e.g., conjugated with biotin or digoxigenin, non-natural amino acids), HCV peptides comprising insertions or deletions (relative to a naturally occurring HCV sequence) of one or more amino acid, as well as fusion proteins. Fusion proteins may be formed between two distinct HCV peptides (see further) or between a HCV peptide and another peptide or protein such as a B-cell epitope, a T-cell epitope, a CTL epitope or a cytokine. Other peptide or protein fusion partners include bovine serum album, keyhole limpet hemocyanin, soybean or horseradish peroxidase, beta-galactosidase, luciferase, alkaline phosphatase, glutathione S-transferase or dihydrofolate reductase or heterologous epitopes such as (histidine)₆-tag, protein A, maltose-binding protein, Tag•100 epitope, c-myc epitope, FLAG®-epitope, lacZ, CMP (calmodulin-binding peptide), HA epitope, protein C epitope or VSV epitope. Other proteins include histones, single-strand binding protein (ssB) and native and engineered fluorescent proteins such as green-, red-, blue-, yellow-, cyan-fluorescent proteins.

The HCV envelope proteins and HCV non-structural proteins correspond to the HCV polyprotein domains spanning amino acids 192-383 (for E1), spanning amino acids 384-809 or 384-746 (for E2-p7 and E2, respectively), spanning amino acids 810-1026 (for NS2), spanning amino acids 1027-1657 (for NS3), spanning amino acids 1658-1711 (for NS4A), spanning amino acids 1712-1972 (for NS4B), spanning amino acids 1973-2420 (for NS5A), and spanning amino acids 2421-3011 (for NS5B). It is to be understood that these protein endpoints are approximations (e.g. the carboxy terminal end of E2 could lie somewhere in the 730-820 amino acid region, e.g. ending at amino acid 730, 735, 740, 742, 744, 745, preferably 746, 747, 748, 750, 760, 770, 780, 790, 800, 809, 810, 820).

In one embodiment, the HCV immunogenic composition, HCV vaccine composition, prophylactic HCV vaccine composition and/or therapeutic HCV vaccine composition of the invention comprise a HCV E1 peptide that is consisting of the HCV polyprotein region spanning amino acids 192 to 326, and an HCV NS3 peptide that is comprising the HCV polyprotein region spanning amino acids 1188 to 1468. More particularly, said HCV NS3 peptide may further comprise the HCV polyprotein region spanning amino acids 1071 to 1084 or parts thereof, such as the HCV polyprotein region spanning amino acids 1073 to 1081. Said HCV NS3 peptide may also comprise the HCV polyprotein region spanning amino acids 1188 to 1468 and the HCV polyprotein region spanning amino acids 1071 to 1084 or parts thereof. In a further embodiment, the HCV immunogenic composition, HCV vaccine composition, prophylactic HCV vaccine composition and/or therapeutic HCV vaccine composition of the invention comprises an HCV E1 peptide defined by SEQ ID NO:1 and an HCV NS3 peptide comprising the HCV polyprotein region spanning amino acids 1188 to 1468 defined by SEQ ID NO:2. More particularly, said HCV NS3 peptide may further comprise the HCV polyprotein region spanning amino acids 1071 to 1084 defined by SEQ ID NO:3 or parts thereof, such as the HCV polyprotein region spanning amino acids 1073 to 1081, defined by, e.g., SEQ ID NO:4. Said HCV NS3 peptide may also comprise the HCV NS3 peptides defined by SEQ ID NO:2 and by SEQ ID NO:3 or SEQ ID NO:4. In particular, such HCV NS3 peptide may be defined by SEQ ID NO:5.

In a further aspect, the current invention relates to an HCV immunogenic composition, an HCV vaccine composition, a prophylactic HCV vaccine composition and/or a therapeutic HCV vaccine composition wherein any of said compositions is comprising, besides the optional pharmaceutically acceptable carrier, at least one HCV envelope peptide and at least one HCV non-structural peptide, and wherein said HCV peptides are linked, optionally via a spacer.

In a further aspect, the current invention relates to an HCV immunogenic composition, an HCV vaccine composition, a prophylactic HCV vaccine composition and/or a therapeutic HCV vaccine composition wherein any of said compositions is comprising, besides the optional pharmaceutically acceptable carrier, at least one HCV envelope peptide and at least one HCV non-structural peptide, and:

wherein said HCV peptides are synthetic peptides or recombinant peptides; and/or

wherein at least one cysteine of said HCV peptides is reversibly or irreversibly blocked; and/or

wherein at least one cysteine of said HCV envelope peptide is alkylated; and/or

wherein at least one cysteine of said HCV non-structural peptide is sulphonated; and/or

wherein said HCV envelope peptide is added to said composition as viral-like particles.

The HCV peptides comprised in the immunogenic composition, vaccine composition, therapeutic vaccine composition or prophylactic vaccine composition according to the present invention may be present as separate, non-linked peptides. Alternatively, said HCV peptides may be linked, optionally via a spacer.

Said linkage may take the form of a spacer-free linear fusion protein wherein two or more peptides are linked via a normal peptide bond involving an alpha amino-group of one peptide and an alpha carboxy-group of another peptide.

Alternatively, a peptide spacer is used to link two peptides. A peptide spacer may be a non-HCV peptide or a HCV peptide not naturally linked to either one of the HCV peptides to be linked. A typical example of such a spacer may be G₄C(G₄S)ₙ or (G₄S)ₙ with n ranging form 1 to 5 (Park et al. 2001, Frankel et al. 2000).

Alternatively, said linkage is taking the form of a branched fusion protein wherein two or more peptides are linked, e.g., via a disulphide bond between naturally and/or non-naturally occurring cysteines, or via a peptide bond involving, e.g., the epsilon amino-group of a naturally or non-naturally occurring lysine present in at least one of said two or more peptides.

It will be clear that branched fusion peptides may be obtained via synthetic means not ruling out recombinant production of the separate peptides and synthetic construction of the branched fusion peptide. Linear fusion peptides, as well as separate non-linked peptides, may be obtained via synthetic means and/or by recombinant production.

Clearly, two or more HCV peptides comprised in the immunogenic composition, vaccine composition, therapeutic vaccine composition or prophylactic vaccine composition according to the present invention may occur linked via a non-peptide spacer such as a “carrier”, e.g., particles of an activated resin capable of covalently or ionically binding a plurality of peptides. Spacers also include particulate compounds or carriers capable of absorbing HCV peptides on their surface and/or in the internal cavities of the particles.

Any of the HCV envelope peptides or HCV nonstructural peptides may, as indicated, be of synthetic origin, i.e. synthesized by applying organic chemistry, or of recombinant origin. HCV peptides may be produced by expression in, e.g., mammalian or insect cells infected with recombinant viruses, yeast cells or bacterial cells.

More particularly, said mammalian cells include HeLa cells, Vero cells, RK13 cells, MRC-5 cells, Chinese hamster ovary (CHO) cells, Baby hamster kidney (BHK) cells and PK15 cells.

More particularly, said insect cells include cells of Spodoptera frugiperda, such as Sf9 cells.

More particularly, said recombinant viruses include recombinant vaccinia viruses, recombinant adenoviruses, recombinant baculoviruses, recombinant canary pox viruses, recombinant Semlike Forest viruses, recombinant alphaviruses, recombinant Ankara Modified viruses and recombinant avipox viruses.

More particularly, said yeast cells include cells of Saccharomyces, such as Saccharomyces cerevisiae, Saccharomyces kluyveri, or Saccharomyces uvarum, Schizosaccharomyces, such as Schizosaccharomyces pombe, Kluyveromyces, such as Kluyveromyces lactis, Yarrowia, such as Yarrowia lipolytica, Hansenula, such as Hansenula polymorpha, Pichia, such as Pichia pastoris, Aspergillus species, Neurospora, such as Neurospora crassa, or Schwanniomyces, such as Schwanniomyces occidentalis, or mutant cells derived from any thereof. More specifically, the HCV peptide or part thereof according to the invention is the product of expression in a Hansenula cell.

More particularly, said bacterial cells include cells of Escherichia coli or Streptomyces species.

In the HCV peptides or parts thereof as described herein, one cysteine residue, or 2 or more cysteine residues comprised in said peptides may be “reversibly or irreversibly blocked”.

An “irreversibly blocked cysteine” is a cysteine of which the cysteine thiol-group is irreversibly protected by chemical or enzymatic means. In particular, “irreversible protection” or “irreversible blocking” by chemical means refers to alkylation, preferably alkylation of a cysteine in a protein by means of alkylating agents, such as, for example, active halogens, ethylenimine or N-(iodoethyl)trifluoro-acetamide. In this respect, it is to be understood that alkylation of cysteine thiol-groups refers to the replacement of the thiol-hydrogen by (CH₂)ₙR, in which n is 0, 1, 2, 3 or 4 and R═H, COOH, NH₂, CONH₂, phenyl, or any derivative thereof. Alkylation can be performed by any method known in the art, such as, for example, active halogens X(CH₂)ₙR in which X is a halogen such as I, Br, Cl or F. Examples of active halogens are methyliodide, iodoacetic acid, iodoacetamide, and 2-bromoethylamine. Other methods of alkylation include the use of NEM (N-ethylmaleimide) or Biotin-NEM or a mixture thereof (Hermanson 1996). The term “alkylating agents” as used herein refers to compounds which are able to perform alkylation as described herein. Such alkylations finally result in a modified cysteine, which can mimic other aminoacids. Alkylation by an ethylenimine results in a structure resembling lysine, in such a way that new cleavage sites for trypsine are introduced (Hermanson 1996). Similarly, the usage of methyliodide results in an amino acid resembling methionine, while the usage of iodoacetate and iodoacetamide results in amino acids resembling glutamic acid and glutamine, respectively. In analogy, these amino acids are preferably used in direct mutation of cysteine.

A “reversibly blocked cysteine” is a cysteine of which the cysteine thiol-groups is reversibly protected. In particular, the term “reversible protection” or “reversible blocking” as used herein contemplates covalently binding of modification agents to the cysteine thiol-groups, as well as manipulating the environment of the protein such, that the redox state of the cysteine thiol-groups remains (shielding). Reversible protection of the cysteine thiol-groups can be carried out chemically or enzymatically.

The term “reversible protection by enzymatical means” as used herein contemplates reversible protection mediated by enzymes, such as for example acyl-transferases, e.g. acyl-transferases that are involved in catalysing thio-esterification, such as palmitoyl acyltransferase.

The term “reversible protection by chemical means” as used herein contemplates reversible protection:

1. by modification agents that reversibly modify cysteinyls such as for example by sulphonation and thio-esterification;

Sulphonation is a reaction where thiol or cysteines involved in disulfide bridges are modified to S-sulfonate: RSH→RS—SO₃⁻ (Darbre 1986) or RS—SR→2RS—SO₃⁻ (sulfitolysis; (Kumar et al. 1986)). Reagents for sulfonation are e.g. Na₂SO₃, or sodium tetrathionate. The latter reagents for sulfonation are used in a concentration of 10-200 mM, and more preferentially in a concentration of 50-200 mM. Optionally sulfonation can be performed in the presence of a catalysator such as, for example Cu²⁺ (100 μM-1 mM) or cysteine (1-10 mM).

The reaction can be performed under protein denaturing as well as native conditions (Kumar et al. 1985, 1986).

Thioester bond formation, or thio-esterification is characterised by:

RSH+R′COX→RS—COR′

in which X is preferentially a halogenide in the compound R′CO—X.

2. by modification agents that reversibly modify the cysteinyls of the present invention such as, for example, by heavy metals, in particular Zn²⁺, Cd²⁺, mono-, dithio- and disulfide-compounds (e.g. aryl- and alkylmethanethiosulfonate, dithiopyridine, dithiomorpholine, dihydrolipoamide, Ellmann reagent, aldrothiol™ (Aldrich) (Rein et al. 1996), dithiocarbamates), or thiolation agents (e.g. gluthathion, N-Acetyl cysteine, cysteineamine). Dithiocarbamate comprise a broad class of molecules possessing an R₁R₂NC(S)SR₃ functional group, which gives them the ability to react with sulphydryl groups. Thiol containing compounds are preferentially used in a concentration of 0.1-50 mM, more preferentially in a concentration of 1-50 mM, and even more preferentially in a concentration of 10-50 mM;

3. by the presence of modification agents that preserve the thiol status (stabilise), in particular antioxidantia, such as for example DTT, dihydroascorbate, vitamins and derivates, mannitol, amino acids, peptides and derivates (e.g. histidine, ergothioneine, carnosine, methionine), gallates, hydroxyanisole, hydoxytoluene, hydroquinon, hydroxymethylphenol and their derivates in concentration range of 10 μM-10 mM, more preferentially in a concentration of 1-10 mM;

4. by thiol stabilising conditions such as, for example, (i) cofactors as metal ions (Zn²⁺, Mg²⁺), ATP, (ii) pH control (e.g. for proteins in most cases pH ˜5 or pH is preferentially thiol pKₐ −2; e.g. for peptides purified by Reversed Phase Chromatography at pH ˜2).

Combinations of reversible protection as described in (1), (2), (3) and (4) may be applied.

The reversible protection and thiol stabilizing compounds may be presented under a monomeric, polymeric or liposomic form.

The removal of the reversibly protection state of the cysteine residues can chemically or enzymatically accomplished by e.g.:

a reductant, in particular DTT, DTE, 2-mercaptoethanol, dithionite, SnCl₂, sodium borohydride, hydroxylamine, TCEP, in particular in a concentration of 1-200 mM, more preferentially in a concentration of 50-200 mM;

removal of the thiol stabilising conditions or agents by e.g. pH increase;

enzymes, in particular thioesterases, glutaredoxine, thioredoxine, in particular in a concentration of 0.01-5 μM, even more particular in a concentration range of 0.1-5 μM.;

combinations of the above described chemical and/or enzymatical conditions.

The removal of the reversibly protection state of the cysteine residues can be carried out in vitro or in vivo, e.g. in a cell or in an individual.

A reductant according to the present invention is any agent which achieves reduction of the sulfur in cysteine residues, e.g. “S—S” disulfide bridges, desulphonation of the cysteine residue (RS—SO₃⁻→RSH). An antioxidant is any reagent which preserves the thiol status or minimises “S—S” formation and/or exchanges. Reduction of the “S—S” disulfide bridges is a chemical reaction whereby the disulfides are reduced to thiol (—SH). Particularly relating to HCV envelope peptides, disulfide bridge breaking agents and methods are disclosed, e.g., by Maertens et al. in International Patent Application Publication No. WO96/04385. “S—S” Reduction can be obtained by (1) enzymatic cascade pathways or by (2) reducing compounds. Enzymes like thioredoxin, glutaredoxin are known to be involved in the in vivo reduction of disulfides and have also been shown to be effective in reducing “S—S” bridges in vitro. Disulfide bonds are rapidly cleaved by reduced thioredoxin at pH 7.0, with an apparent second order rate that is around 104 times larger than the corresponding rate constant for the reaction with DTT. The reduction kinetic can be dramatically increased by preincubation the protein solution with 1 mM DTT or dihydrolipoamide (Holmgren 1979). Thiol compounds able to reduce protein disulfide bridges are for instance Dithiothreitol (DTT), Dithioerythritol (DTE), β-mercaptoethanol, thiocarbamates, bis(2-mercaptoethyl) sulfone and N,N′-bis(mercaptoacetyl)hydrazine, and sodium-dithionite. Reducing agents without thiol groups like ascorbate or stannous chloride (SnCl₂), which have been shown to be very useful in the reduction of disulfide bridges in monoclonal antibodies (Thakur et al. 1991), may also be used for the reduction of HCV proteins. In addition, changes in pH values may influence the redox status of HCV proteins. Sodium borohydride treatment has been shown to be effective for the reduction of disulfide bridges in peptides (Gailit 1993). Tris (2-carboxyethyl)phosphine (TCEP) is able to reduce disulfides at low pH (Burns et al. 1991). Selenol catalyses the reduction of disulfide to thiols when DTT or sodium borohydride is used as reductant. Selenocysteamine, a commercially available diselenide, was used as precursor of the catalyst (Singh & Kats 1995).

The terms “virus-like particle”, “viral-like particle”, or “VLP” is herein defined as structures of a specific nature and shape containing several basic units of the HCV E1 and/or E2 envelope proteins, which on their own are thought to consist of one or two E1 and/or E2 monomers, respectively. It should be clear that the particles of the present invention are defined to be devoid of infectious HCV RNA genomes. The particles of the present invention can be higher-order particles of spherical nature which can be empty, consisting of a shell of envelope proteins in which lipids, detergents, the HCV core protein, or adjuvant molecules can be incorporated. The latter particles can also be encapsulated by liposomes or apolipoproteins, such as, for example, apolipoprotein B or low density lipoproteins, or by any other means of targeting said particles to a specific organ or tissue. In this case, such empty spherical particles are often referred to as “virus-like particles” or VLPs. Alternatively, the higher-order particles can be solid spherical structures, in which the complete sphere consists of HCV E1 or E2 envelope protein oligomers, in which lipids, detergents, the HCV core protein, or adjuvant molecules can be additionally incorporated, or which in turn may be themselves encapsulated by liposomes or apolipoproteins, such as, for example, apolipoprotein B, low density lipoproteins, or by any other means of targeting said particles to a specific organ or tissue, e.g. asialoglycoproteins. The particles can also consist of smaller structures (compared to the empty or solid spherical structures indicated above) which are usually round (see further)-shaped and which usually do not contain more than a single layer of HCV envelope proteins. A typical example of such smaller particles are rosette-like structures which consist of a lower number of HCV envelope proteins, usually between 4 and 16. A specific example of the latter includes the smaller particles obtained with E1s in 0.2% CHAPS as exemplified herein which apparently contain 8-10 monomers of E1s. Such rosette-like structures are usually organized in a plane and are round-shaped, e.g. in the form of a wheel. Again lipids, detergents, the HCV core protein, or adjuvant molecules can be additionally incorporated, or the smaller particles may be encapsulated by liposomes or apolipoproteins, such as, for example, apolipoprotein B or low density lipoproteins, or by any other means of targeting said particles to a specific organ or tissue. Smaller particles may also form small spherical or globular structures consisting of a similar smaller number of HCV E1 or E2 envelope proteins in which lipids, detergents, the HCV core protein, or adjuvant molecules could be additionally incorporated, or which in turn may be encapsulated by liposomes or apolipoproteins, such as, for example, apolipoprotein B or low density lipoproteins, or by any other means of targeting said particles to a specific organ or tissue. The size (i.e. the diameter) of the above-defined particles, as measured by the well-known-in-the-art dynamic light scattering techniques, is usually between 1 to 100 nm, more preferentially between 2 to 70 nm. Virus-like particles of HCV envelope proteins have been described in International Patent Application Publication Nos. WO99/67285, WO02/055548 and in International Patent Application No. PCT/BE02/00063.

In another aspect, the current invention relates to an HCV immunogenic composition, an HCV vaccine composition, a prophylactic HCV vaccine composition and/or a therapeutic HCV vaccine composition wherein any of said compositions is comprising, besides the optional pharmaceutically acceptable carrier:

a plurality of HCV envelope peptides derived from different HCV genotypes, subtypes or isolates and at least one HCV non-structural peptide; or

at least one HCV envelope peptide and a plurality of HCV non-structural peptides derived from different HCV genotypes, subtypes or isolates; or

a plurality of HCV envelope peptides derived from different HCV genotypes, subtypes or isolates and a plurality of HCV non-structural peptides derived from different HCV genotypes, subtypes or isolates.

Currently known HCV types include HCV genotypes 1, 2, 3, 4, 5, 6, 7, 8, 9, 10, 11 and known subtypes thereof include HCV subtypes 1a, 1b, 1c, 1d, 1e, 1f, 1g, 2a, 2b, 2c, 2d, 2e, 2f, 2g, 2h, 2i, 2k, 2l, 3a, 3b, 3c, 3d, 3e, 3f, 3g, 4a, 4b, 4c, 4d, 4e, 4f, 4g, 4h, 4i, 4j, 4k, 4l, 4m, 5a, 6a, 6b, 7a, 7b, 7c, 7d, 8a, 8b, 8c, 8d, 9a, 9b, 9c, 10a and 11a. The sequences of cDNA clones covering the complete genome of several prototype isolates have been determined and include complete prototype genomes of the HCV genotypes 1a (e.g., GenBank accession number AF009606), 1b (e.g., GenBank accession number AB016785), 1c (e.g., GenBank accession number D14853), 2a (e.g., GenBank accession number AB047639), 2b (e.g., GenBank accession number AB030907), 2c (e.g., GenBank accession number D50409) 2k (e.g., GenBank accession number AB031663), 3a (e.g., GenBank accession number AF046866), 3b (e.g., GenBank accession number D49374), 4a (e.g., GenBank accession number Y11604), 5a (e.g., GenBank accession number AF064490), 6a (e.g., GenBank accession number Y12083), 6b (e.g., GenBank accession number D84262), 7b (e.g., GenBank accession number D84263), 8b (e.g., GenBank accession number D84264), 9a (e.g., GenBank accession number D84265), 10a (e.g., GenBank accession number D63821) and 11a (e.g., GenBank accession number D63822). A new HCV genotype was further described in International Patent Application No. PCT/EP02/09731. An HCV isolate is to be considered as a HCV quasispecies isolated from a HCV-infected mammal. A HCV quasispecies usually comprises a number of variant viruses with variant genomes usually of the same HCV type or HCV subtype.

Further aspects of the current invention comprise the use of an HCV immunogenic composition, an HCV vaccine composition, a prophylactic HCV vaccine composition and/or a therapeutic HCV vaccine composition according to the invention for:

inducing in a mammal a humoral response to the HCV peptides comprised in any of said composition; and/or

inducing in a mammal a cellular response to the HCV peptides comprised in any of said composition, wherein said cellular response may be a CD4⁺ T-cell proliferation response and/or a CD8⁺ cytotoxic T-cell response and/or the increased production of cytokines; and/or

for prophylactic protection of a mammal against chronic HCV infection, wherein said HCV may be a homologous or a heterologous HCV; and/or

for therapeutically treating a chronically HCV-infected mammal, wherein said HCV may be a homologous or a heterologous HCV; and/or

for reducing liver disease in a HCV-infected mammal; and/or

for reducing liver disease in a chronic HCV-infected mammal by at least 2 points according to the overall Ishak score; and/or

for reducing serum liver enzyme activity levels in a HCV-infected mammal, wherein said liver enzyme may be, e.g., alanine aminotransferase (ALT) or gamma-glutamylpeptidase; and/or

for reducing HCV RNA levels in a HCV-infected mammal; or

for reducing liver fibrosis progression in a HCV-infected mammal; and/or

for reducing liver fibrosis in a HCV-infected mammal.

Said mammal obviously may be a human.

In particular, the uses according to the invention are methods for obtaining at least one of the recited effects, with said methods comprising administering any of said compositions to a mammal or a human.

An epitope is referring to a structure capable of binding to and/or activating a cell involved in eliciting an immune response to said structure. Epitopes thus include epitopes of B-cells, T-cells, T-helper cells and CTLs. Epitopes include conformational epitopes and linear epitopes. A linear epitope is a limited set of, e.g., contiguous elements of a repetitive structure construed with a limited number of distinct elements. A conformational epitope usually comprises, e.g., discontigous elements of such a repetitive structure which are, however, in close vicinity due to the three-dimensional folding of said repetitive structure. A well-known example of such a repetitive structure is a peptide or protein wherein the contiguous or discontiguous elements are amino acids. Peptide- or protein-epitopes comprise peptides or parts of peptides or proteins capable of binding to, e.g., T-cell receptors, B-cell receptors, antibodies or MHC molecules. The size of linear peptide- or protein-epitopes can be limited to a few, e.g. 4, 5, 6, 7, 8, 9, 10, 11, 12, 13, 14 or 15 amino acids. An epitope is antigenic but not always immunogenic.

A T-cell stimulating epitope refers to an epitope capable of stimulating T-cells, T-helper cells or CTL-cells. A T-helper cell stimulating epitope may be selected by monitoring the lymphoproliferative response, also referred to as CD4⁺ T-cell proliferation response, towards (potential antigenic) polypeptides containing in their amino acid sequence a (putative) T-cell stimulating epitope. Said lymphoproliferative response may be measured by either a T-helper assay comprising in vitro stimulation of peripheral blood mononuclear cells (PBMCs) from patient sera with varying concentrations of peptides to be tested for T-cell stimulating activity and counting the amount of radiolabelled thymidine taken up by the PBMCs. Proliferation is considered positive when the stimulation index (mean cpm of antigen-stimulated cultures/mean cpm of controle cultures) is more than 1, preferably more than 2, most preferably more than 3. A CTL-stimulating epitope may be selected by means of a cytotoxic T-lymphocyte or cytotoxic T-cell (CTL) assay measuring the lytic activity of cytotoxic cells, also referred to as CD8⁺ CTL response, using ⁵¹Cr release. Cell-mediated responses may also be assessed by measuring cytokine production, e.g., by an ELISpot assay (see for instance Fujihashi et al. 1993). Characteristic for a Th1-like response is the production/secretion of, e.g., IL-2 and/or IFN-γ. Characteristic for a Th2-like response is the production/secretion of, e.g., IL-4.

With “prophylactic protection against infection by a homologous HCV” is meant that protection is obtained against a challenge HCV virus of exactly the same genotype, subtype or isolate as compared to the HCV genotype, subtype or isolate from which the HCV antigen or HCV antigens are derived. A composition may for example comprise a HCV envelope peptide and a peptide of a HCV non-structural protein both of which are derived from a particular HCV type 1b isolate. A “homologous HCV” would in this case be the same particular HCV type 1b isolate. “Homologous” in the context of “therapeutic treatment of a HCV homologous to the HCV peptides in a composition” has to be interpreted likewise.

With “prophylactic protection against infection by a heterologous HCV” is meant that protection is obtained against a challenge HCV virus classified in another genotype, subtype, or isolate as compared to the HCV genotype, subtype or isolate from which the HCV antigen or HCV antigens are derived. A composition may for example comprise a HCV envelope peptide and a peptide of a HCV non-structural protein both of which are derived from a HCV type 1b isolate. A “heterologous HCV” would in this case be, e.g., a HCV type. 1b isolate sufficiently different from the type 1b isolate from which the antigens were derived, a type 1a HCV virus or a type 7 HCV virus. “Sufficiently different” as used in this particular context is to be understood at least a difference of 2%, 3% or 4% on the amino acid level. “Heterologous” in the context of “therapeutic treatment of a HCV heterologous to the HCV peptides in a composition” has to be interpreted likewise.

With the term “liver disease” is meant in this context any abnormal liver condition caused by infection with the hepatitis C virus including inflammation, fibrosis, cirrhosis, necrosis, necro-inflammation and hepatocellular carcinoma.

With “reducing liver disease” is meant any stabilization or reduction of the liver disease status. Liver disease can be determined, e.g., by the Knodell scoring system (Knodell et al. 1981) or the Knodell scoring system adapted by Ishak (Ishak et al. 1995). A reduction of this score by two points is accepted as therapeutically beneficial effect in several studies (see, e.g., studies published after 1996 as indicated in Table 2 of Shiffman 1999).

With “reducing liver fibrosis progression” is meant any slowing down, halting or reverting of the normally expected progression of liver fibrosis. Liver fibrosis progression can be determined, e.g., by the Metavir scoring system. Normal expected progression of liver fibrosis according to this system was published to be an increase of the Metavir score of an untreated chronic HCV patient of approximately 0.133 per year (Poynard et al. 1997). “Reducing liver fibrosis” is meant to comprise any reduction of the normally expected progression of liver fibrosis.

Liver fibrosis and inflammation can be scored according to the Ishak scoring system (which is a modification of the scoring system of Knodell et al. 1981; Ishak et al. 1995) or Metavir scoring system (Bedossa and Poynard 1996). The Ishak scores range from 0 to 18 for grading of inflammation and from 0 to 6 for staging of fibrosis/cirrhosis. The sum of the Ishak inflammation and fibrosis scores comes closest to the Histological Activity Index (HAI; Knodell et al. 1981) which has been widely used. The Metavir scores range from 0 to 3 for grading of inflammation and from 0 to 4 for staging of fibrosis/cirrhosis. The overall progression rate of the Metavir score in an untreated patient is estimated to be 0.133 per year (Poynard et al. 1997).

Other aspects of the invention relate to methods of vaccinating a HCV-naïve or HCV-infected mammal comprising administering a DNA vaccine and an HCV immunogenic composition, an HCV vaccine composition, a prophylactic HCV vaccine composition and/or a therapeutic HCV vaccine composition according to the invention.

The immunogenic composition, vaccine composition, therapeutic vaccine composition or prophylactic vaccine composition as described above may in addition comprise DNA vectors wherein said DNA vectors are capable of effectuating expression of an antigen. Particularly relating to the current invention, the HCV immunogenic composition, HCV vaccine composition, therapeutic HCV vaccine composition or prophylactic HCV vaccine composition may in addition comprise DNA vectors wherein said DNA vectors are capable of effectuating expression of one or more HCV envelope peptide and/or of one or more HCV nonstructural peptide. Alternatively, the protein- or peptide-based immunogenic composition, vaccine composition, therapeutic vaccine composition or prophylactic vaccine composition of the invention may be used in combination with a DNA vector-based immunogenic composition, vaccine composition, therapeutic vaccine composition or prophylactic vaccine composition (also referred to as “DNA vaccine”). Such combination for instance includes a DNA-prime protein-boost vaccination scheme wherein vaccination is initiated by administering a DNA vector-based immunogenic composition, vaccine composition, therapeutic vaccine composition or prophylactic vaccine composition and is followed by administering a protein- or peptide-based immunogenic composition, vaccine composition, therapeutic vaccine composition or prophylactic vaccine composition of the invention. In particular the DNA vector is capable of expressing one or more HCV antigens.

With a “DNA vector” is meant any DNA carrier comprising the open reading frame for one or more of the peptides useful for eliciting and/or enhancing an immune response. In general, said open reading frames are operably linked to transcription regulatory elements, such as promoters and terminators, enabling expression of the peptide encoded by the open reading frame. The term “DNA vector” is meant to include naked plasmid DNA, plasmid DNA formulated with a suitable pharmaceutically acceptable carrier, recombinant viruses (e.g., as described above), or recombinant viruses formulated with a suitable pharmaceutically acceptable carrier.

As used herein, the term “transcription regulatory elements” refers to a nucleotide sequence which contains essential regulatory elements, such that upon introduction into a living vertebrate cell it is able to direct the cellular machinery to produce translation products encoded by the polynucleotide.

The term “operably linked” refers to a juxtaposition wherein the components are configured so as to perform their usual function. Thus, transcription regulatory elements operably linked to a nucleotide sequence are capable of effecting the expression of said nucleotide sequence. Those skilled in the art can appreciate that different transcriptional promoters, terminators, carrier vectors or specific gene sequences may be used successfully.

A “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. Preferably, a pharmaceutically acceptable carrier or adjuvant enhances the immune response elicited by an antigen. Suitable carriers or adjuvantia typically comprise one or more of the compounds included in the following non-exhaustive list:

large slowly metabolized macromolecules such as proteins, polysaccharides, polylactic acids, polyglycolic acids, polymeric amino acids, amino acid copolymers and inactive virus particles;

aluminium hydroxide, aluminium in combination with 3-O-deacylated monophosphoryl lipid A (see International Patent Application Publication No. WO93/19780), or aluminium phosphate (see International Patent Application Publication No. WO93/24148);

N-acetyl-muramyl-L-threonyl-D-isoglutamine (see U.S. Pat. No. 4,606,918), N-acetyl-normuramyl-L-alanyl-D-isoglutamine, N-acetylmuramyl-L-alanyl-D-isoglutamyl-L-alanine2-(1′,2′-dipalmitoyl-sn-glycero-3-hydroxyphosphoryloxy) ethyl amine;

RIBI (ImmunoChem Research Inc., Hamilton, Mont., USA) which contains monophosphoryl lipid A (i.e., a detoxified endotoxin), trehalose-6,6-dimycolate, and cell wall skeleton (MPL+TDM+CWS) in a 2% squalene/Tween 80 emulsion. Any of the three components MPL, TDM or CWS may also be used alone or combined 2 by 2. The MPL may also be replaced by its synthetic analogue referred to as RC-529 or by any other amino-alkyl glucosaminide 4-phosphate (Johnson et al. 1999, Persing et al. 2002);

adjuvants such as Stimulon (Cambridge Bioscience, Worcester, Mass., USA), SAF-1 (Syntex);

bacterial DNA-based adjuvants such as ISS (Dynavax) or CpG (Coley Pharmaceuticals);

adjuvants such as combinations between QS21 and 3-de-O-acetylated monophosphoryl lipid A (see International Patent Application Publication No. WO94/00153) which may be further supplemented with an oil-in-water emulsion (see, e.g., International Patent Application Publication Nos. WO95/17210, WO97/01640 and WO9856414) in which the oil-in-water emulsion comprises a metabolisable oil and a saponin, or a metabolisable oil, a saponin, and a sterol, or which may be further supplemented with a cytokine (see International Patent Application Publication No. WO98/57659);

adjuvants such as MF-59 (Chiron), or poly[di(carboxylatophenoxy) phosphazene] based adjuvants (Virus Research Institute);

blockcopolymer based adjuvants such as Optivax (Vaxcel, Cythx) or inulin-based adjuvants, such as Algammulin and GammaInulin (Anutech);

Complete or Incomplete Freund's Adjuvant (CFA or IFA, respectively) or Gerbu preparations (Gerbu Biotechnik). It is to be understood that Complete Freund's Adjuvant (CFA) may be used for non-human applications and research purposes as well;

a saponin such as QuilA, a purified saponin such as QS21, QS7 or QS17, β-escin or digitonin;

immunostimulatory oligonucleotides comprising unmethylated CpG dinucleotides such as [purine-purine-CG-pyrimidine-pyrimidine] oligonucleotides. Immunostimulatory oligonucleotides may also be combined with cationic peptides as described, e.g., by Riedl et al. (2002);

Immune Stimulating Complexes together with saponins, for example Quil A (ISCOMS);

excipients and diluents, which are inherently non-toxic and non-therapeutic, such as water, saline, glycerol, ethanol, wetting or emulsifying agents, pH buffering substances, preservatives, and the like;

a biodegradable and/or biocompatible oil such as squalane, squalene, eicosane, tetratetracontane, glycerol, peanut oil, vegetable oil, in a concentration of, e.g., 1 to 10% or 2.5 to 5%;

vitamins such as vitamin C (ascorbic acid or its salts or esters), vitamin E (tocopherol), or vitamin A;

carotenoids, or natural or synthetic flavanoids;

trace elements, such as selenium.

Any of the afore-mentioned adjuvants comprising 3-de-O-acetylated monophosphoryl lipid A, said 3-de-O-acetylated monophosphoryl lipid A may be forming a small particle (see International Patent Application Publication No. WO94/21292).

Typically, a vaccine composition is prepared as an injectable, either as a liquid solution or suspension. Injection may be subcutaneous, intramuscular, intravenous, intraperitoneal, intrathecal, intradermal, intraepidermal. Other types of administration comprise implantation, suppositories, oral ingestion, enteric application, inhalation, aerosolization or nasal spray or drops. Solid forms, suitable for solution on, or suspension in, liquid vehicles prior to injection may also be prepared. The preparation may also be emulsified or encapsulated in liposomes for enhancing adjuvant effect. The polypeptides may also be incorporated into.

### EXAMPLES

### Example 1

### Production of HCV E1s in Yeast

The HCV E1s protein (amino acids 192-326 of the HCV polyprotein; SEQ ID NO:1) was purified from a precursor protein expressed in Hansenula polymorpha RB11 cells. Said precursor protein comprised a chicken lysozyme leader (CL), a his-tag (H6) and a lysine (K) at the N-terminal end of the mature HCV E1s protein (CL-H6-K-E1s).

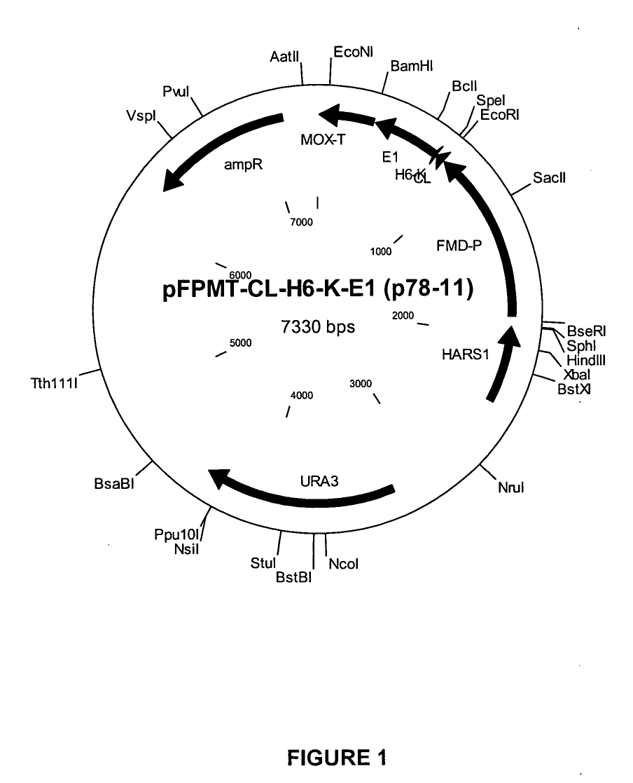

Construction of the shuttle vector pFPMT-CL-H6-K-E1s (schematically drawn in FIG. 1) is described in Example 5 of International Patent Application Publication No. WO02/086100.

Transformation of H. polymorpha RB11 with pFPMT-CL-H6-K-E1s and selection of transformants are described in Example 6 of International Patent Application Publication No. WO02/086100.

Fermentation conditions for expression of the HCV E1s protein by H. polymorpha transformed with pFPMT-CL-H6-K-E1s is described in Example 14 of International Patent Application Publication No. WO02/086100.

Purification, including removal of the H6-K-tag, of mature HCV E1s (alkylated with iodoacetamide) from the H6-K-E1s precursor protein expressed in H. polymorpha RB11 transformed with pFPMT-CL-H6-K-E1s is described in Example 18 of International Patent Application Publication No. WO02/086100 (referring back in part to Example 17 of International Patent Application Publication No. WO02/086100).

Formation of viral-like particles (VLPs) in PBS, 0.5% (w/v) betain with the purified HCV E1s protein (alkylated with iodoacetamide; at a concentration of 400 μg/mL) is described in Example 20 of International Patent Application Publication No. WO02/086100.

### Example 2

### Formulation of HCV E1s Vaccine Composition

Starting material was the composition of HCV E1s viral-like particles obtained as described in Example 1 (in PBS, 0.5% (w/v) betain and at an E1s concentration of 400 μg/ml). Equal volumes of this E1s VLP-composition and of Alhydrogel 1.3% (Superfos, Denmark) were mixed. The resulting mix was finally further diluted with 19 volumes of 0.9% NaCl to yield alum-adjuvanted E1s at a concentration of 20 μg E1s/mL and 0.065% of Alhydrogel.

### Example 3

### Production of E1s from Vero Cells

The HCV E1s protein (amino acids 192-326 of the HCV polyprotein; the same mature E1s as described in Example 1) was expressed in Vero cells using recombinant vaccinia virus HCV11B. This vaccinia virus is essentially identical to vvHCV11A (as described in U.S. Pat. No. 6,150,134) but has been passaged from RK13 to Vero cells. The E1s protein was purified (by means of lentil chromatography, reduction-alkylation and size exclusion chromatography) essentially as described in Example 5 of U.S. Pat. No. 6,150,134 but modified according to Example 9 of International Patent Application No. PCT/EP99/04342 (Publication No. WO99/67285), making use of iodoacetamide (instead of N-ethyl maleimide) as alkylating agent for the cysteines. After purification the 3% Empigen-BB was exchanged for 3% betain by size exclusion chromatography as described in Example 1 of International Patent Application No. PCT/EP99/04342 (Publication No. WO99/67285). This process allows recovery of E1s as a viral-like particle. Finally the material was desalted to PBS containing 0.5% betain and an E1s concentration of 400 μg/mL. This E1s was mixed with an equal volume of Alhydrogel 1.3% (Superfos, Denmark) and finally further diluted with 19 volumes of 0.9% NaCl to yield alum-adjuvanted E1s at a concentration of 20 μg E1s/mL and 0.065% of Alhydrogel.

### Example 4

### Production of NS3 in Escherichia coli

The HCV NS3-TN protein (amino acids 1166-1468 of the HCV polyprotein in which the amino acids 1167 to 1180 have been replaced by the amino acids 1071-1084 and in which amino acid 1166 was mutated into a methionine, as described in Example 7a of International Patent Application No. PCT/EP99/04342 (Publication No. WO 99/67285); SEQ ID NO:5) was expressed in E. Coli. The protein was purified essentially as described in Example 7b of International Patent Application No. PCT/EP99/04342 (Publication No. WO 99/67285), making use of sulfonation as modifying agent for the cysteines. Finally the material was desalted to PBS, pH 7.5 containing 6 M urea and an NS3-TN protein concentration of 1.3 mg/mL. This NS3 was after a dilution to 400 μg/mL with 0.9% NaCl, mixed with an equal volume of Alhydrogel 1.3% (Superfos, Denmark) and finally further diluted with 19 volumes of 0.9% NaCl to yield alum-adjuvanted NS3 at a concentration of 20 μg NS3/mL and 0.065% of Alhydrogel.

### Example 5

### Immunogenicity Study in Rhesus Monkeys

The housing, maintenance, and care of the animals were in compliance with all relevant guidelines and requirements.

Eight (8) rhesus monkeys (Macaca mulatta) were intramuscularly vaccinated with a dose of 10 μg NS3-TN in the upper right limb. Half of these animals were also vaccinated with a dose of 10 μg E1s from Vero-cells and the other half of the animals received 10 μg E1s from yeast.

The E1s-vaccines were administered in the upper left limb. As described in Examples 1-3 all proteins were formulated on alum. The animals received immunizations at week 0, 3 and 9 and the immune response was assessed 2 weeks after the third immunization (i.e. at week 11).

Antibody Titres

Antibody titers were determined by ELISA. A serial dilution of a serum sample was compared to an in house standard (this in house standard defined as having 1000 mU/mL of E1s antibody is a mixture of three sera from HCV chronic carriers selected based on a high anti-envelope titer). The detection limit for this assay is 5 mU/ml.

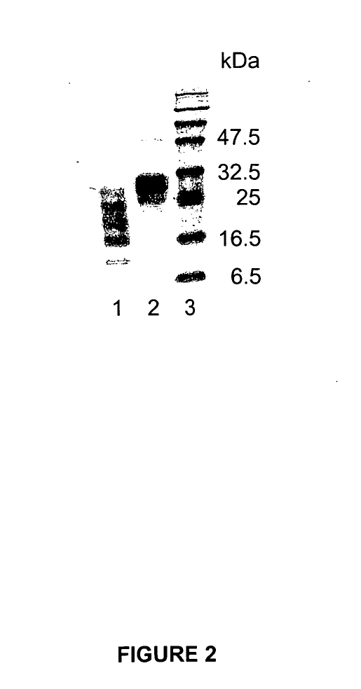

All animals mounted an antibody response against the proteins used for immunization. The level of antibody, expressed of log(mU/mL)+/−SD (standard deviation) was similar to the level as found in the standard which is based on carriers with high level of anti-E1s antibody, this both for the animals immunized with the yeast- and Vero-derived E1s. The similarity of these results with E1s obtained from a yeast (H. polymorpha) and with E1s obtained from a mammalian (Vero) cell line are surprising taking in account the large difference in biochemical parameters between both molecules. The yeast E1s protein is composed of a ladder of differently glycosylated forms of E1s while the Vero-derived E1s is composed of a single band of protein which is homogeneously glycosylated (illustrated in FIG. 2). Overall there is even a tendency for the yeast-derived E1s protein inducing a higher response than the response obtained with the Vero cell-derived E1s.

TABLE 1
Antibodies induced in rhesus monkey upon
immunization with E1s from yeast or from
Vero cells (expressed as log (mU/mL)).

| | ELISA with yeast E1s | ELISA with Vero E1s |
|---|---|---|
| Monkeys immunized | 2.85 +/− 0.32 | 3.06 +/− 0.29 |
| with Vero | | |
| E1s (n = 4) | | |
| Monkeys immunized | 3.32 +/− 0.22 | 3.39 +/− 0.2 |
| with yeast | | |
| E1s (n = 4) | | |

Antibody responses to NS3 were determined in a similar way. A mean titer expressed as log (mU/mL)+/−SD of 2.74+/−0.32 (n=8) was reached which is again of same order of an NS3 response observed in chronic carriers and which shows that the NS3 protein is immunogenic.

T-Cell Immunity

Peripheral blood mononuclear cells (PBMC), isolated from blood drawn at week 0, or at week 11, at a concentration of 4×10⁵ cells/well in a total volume of 200 μL were cultured in complete RPMI-1640 medium in U-shaped 96-well microtitre plates, together with either ConA (5 μg/mL, positive control), or recombinant yeast E1s for the animals immunized with yeast E1s, or Vero-E1s for the animals immunized with Vero-E1s, or NS3 proteins (all at 5 μg/mL) or with medium alone (negative control) for 90 h at 37 C in a humidified atmosphere containing 5% CO₂. A series of experiments was performed to establish the most appropriate incubation time for both the mitogen- and antigen-induced proliferative response. Culturing the cells for 90 h (including the time for ³H-thymidine uptake) was found to be sufficient for mitogenic stimulation as well as for antigen-induced responses. During the last 18 hours, the cells were pulsed with 2 μCi (3H-TRK758) thymidine per well. Subsequently, the cultures were harvested on glass fiber filters and label uptake is determined by counting simultaneously in an Packard Top Counter (Direct Beta Counter).

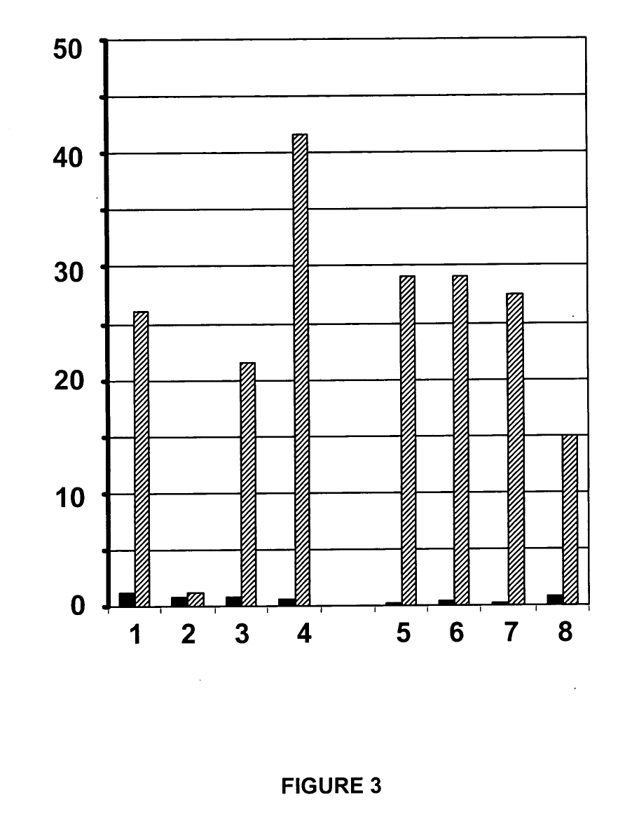

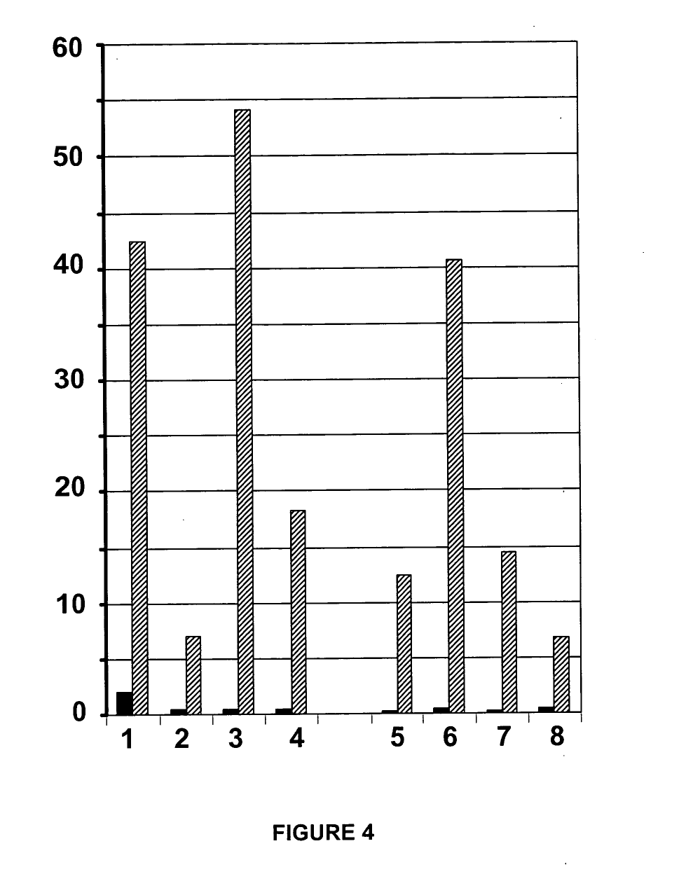

Results are expressed as the stimulation index (SI), which is the ratio of thymidine incorporated in the cells cultured with envelope antigen versus the ones cultured without antigen. A stimulation index of >3 is considered a positive signal. All animals did react in a satisfactory way to ConA proving the quality of the cells used in the assay. From the results shown in FIG. 3, it can be concluded that for E1s, 7 out of the 8 animals had a clear cut antigen-specific proliferation at week 11 which was absent at week 0. For NS3, all 8 animals did mount such a response (FIG. 4). The high level of T-cell proliferation for both E1s and NS3 was surprising since the alum-adjuvant used is mainly known to stimulate humoral immune responses. This clearly demonstrates the high immunogenic potential of both E1s and NS3 in stimulating T-cells in the same single individual.

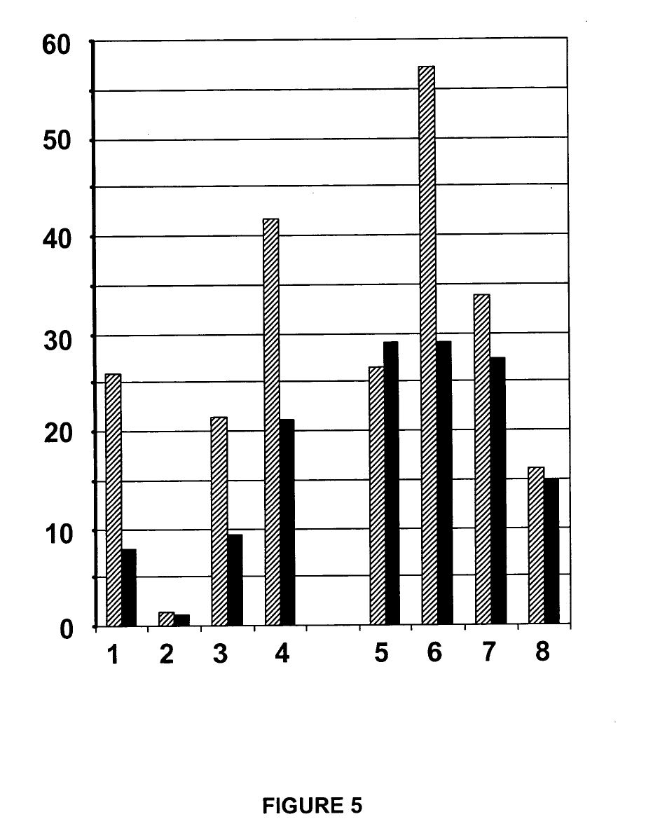

In addition a control experiment was performed in which PBMC from all E1s-immunized animals were restimulated with E1s from yeast and E1s from Vero cells, this in order to establish the cross-reactivity of both antigens. This experiment (results presented in FIG. 5) confirmed that indeed 7 out of the 8 μl s-immunized animals did mount a high T-cell response against E1s and that all of the 7 animals reacted both to the yeast- and Vero-derived E1s material irrespective of the antigen used for vaccination.

This is the first demonstration in a higher mammalian species of a combination of an HCV envelope antigen and a HCV non-structural antigen formulated on the same adjuvant and administered in a single animal which is resulting in a high and specific immune response. Both the humoral and cellular compartment of the immune system were activated when using this combination. The good cross-reactivity as observed between E1s derived from yeast and from mammalian cells is supportive for bio-equivalence of both materials and is suprising based on the significant difference on a biophysical level between both products. Therefore the yeast-E1s should be able to replace the mammalian-E1s which is known to induce a protective immunization response against chronic disease in chimpanzee upon challenge infection (as described in International Patent Application No. PCT/EP02/00219, published as WO02/055548). The demonstration that NS3, and more specifically NS3 formulated with the same adjuvant as E1s, induces significant T-cell responses is a clear indication that combining E1s with NS3 broadens the HCV specific immune response and will be helpful in controlling HCV infection even more efficiently.

### Example 6

### Prophylactic Protective Immunization of Chimpanzees Vaccinated with a Combination of E1s and NS3

The housing, maintenance, and care of the animals were in compliance with all relevant guidelines and requirements. H. polymorpha-derived E1s and E. coli-derived NS3-TN were formulated on alum, yielding a final formulation of 40 μg of E1s/mL or NS3/mL and 0.13% of alum. Three chimpanzees (Pan troglodytes) were immunized intramuscularly with 1.25 mL E1s (left upper limb) and 1.25 mL NS3 (right upper limb). A fourth chimpanzee was immunized simultaneously in both upper limbs with 1.25 mL of placebo consisting of 0.13% alum only. Immunizations were performed at weeks 0, 4, 8 and 20. Finally, the animals were challenged at week 24 with 100 CID50 (chimp infectious doses) of the inoculum J4.91 provided by Dr. R. Purcell (Hepatitis Virus Section, NIH, Bethesda, Md.). Viremia levels in the serum of challenged chimpanzees are analyzed using Roche Monitor HCV during 12 months post challenge. Animals with undetectable viremia are classified as fully protected, animals resolving viremia no later than 6 months after challenge and without a rebound of viral RNA within the 6 following months are classified as acute resolving while animals still viremic after 6 months are classified as chronically infected.

After completion of the immunization schedule for these four chimpanzees, antibody titers against E1 and NS3 were determined. For E1, antibody titers both against yeast- and Vero-derived were determined using ELISA. For NS3 antibody titers both against the sulphonated and desulphonated protein were determined using ELISA. Desulphonation was performed by incubating the sulphonated NS3 with 5 mM of DTT during the coating time (3 μg/ml of NS3, 1 hour at 37° C.) of the ELISA plates. Titers were defined as the dilution of the serum still yielding an OD twice as high as the background of the assay. The results are summarized in Table 2.

The titration results using E1 from yeast or Vero were very similar. More importantly the titers were very similar to those obtained in chimpanzees immunized with E1-Vero (as described in Example 15 of International Patent Application WO02/055548) from which few samples were titrated again using the same assay as for the 4 chimpanzees of this study. Taking into account that the chimpanzees in this study only received 4 immunizations while the historical animals received 6 immunizations, this confirms again that the yeast-derived E1 has an equivalent or even superior immunogenicity compared to the Vero-derived E1.

Surprisingly, the NS3 responses measured with the desulphonated protein were much higher than the ones measured with sulphonated protein. This result may be important as this indicates that NS3 is desulphonated in vivo, prior to induction of the immune response. Especially for T-cell responses this may be very important as the T-cell must be able to recognize the native NS3 which is not sulphonated.

TABLE 2
Overview of antibody titers induced by vaccination of 4
chimpanzees and comparison with 3 historical animals from
another study (as described in Example 15 of WO02/055548).
Titers have been measured for E1 both against the yeast-and
Vero-derived E1 and for NS3 against sulfonated (SO₃)
or desulfonated (DS) proteins.

| | | | | | | |
|---|---|---|---|---|---|---|
| | | | E1 | E1 | NS3- | NS3- |
| | | | yeast | Vero | SO₃ | DS |
| Chimp | Immunization | # Immunizations received | Titer | Titer | Titer | Titer |

This study

| | | | | | | |
|---|---|---|---|---|---|---|
| CH 5835 | E1 yeast + NS3 | pre | <200 | <200 | <200 | <200 |
| | E. coli | pre | <200 | <200 | <200 | <200 |
| | | pre | <200 | <200 | <200 | <200 |
| | | just after/before 1^{st} | <200 | <200 | <200 | <200 |
| | | 2 weeks after 3^{rd} | 17788 | 16946 | 3091 | 18921 |
| | | 2 weeks after boost (4^{th}) = | 44031 | 34199 | 4975 | 33720 |
| | | 2 weeks before challenge | | | | |
| CH 5855 | Placebo | pre | <200 | <200 | <200 | <200 |
| | | pre | <200 | <200 | <200 | <200 |
| | | pre | <200 | <200 | <200 | <200 |
| | | just after/before 1^{st} | <200 | <200 | <200 | <200 |
| | | 2 weeks after 3^{rd} | <200 | <200 | <200 | <200 |
| | | 2 weeks after boost (4^{th}) = | <200 | <200 | <200 | <200 |
| | | 2 weeks before challenge | | | | |
| CH 5872 | E1 yeast + NS3 | pre | <200 | <200 | <200 | <200 |
| | E. coli | pre | <200 | <200 | <200 | <200 |
| | | pre | <200 | <200 | <200 | <200 |
| | | just after/before 1^{st} | <200 | <200 | <200 | <200 |
| | | 2 weeks after 3^{rd} | 44348 | 34648 | 4008 | 15912 |
| | | 2 weeks after boost (4^{th}) = | 48952 | 42069 | 5525 | 19485 |
| | | 2 weeks before challenge | | | | |
| CH 5960 | E1 yeast + NS3 | pre | <200 | <200 | <200 | <200 |
| | E. coli | pre | <200 | <200 | <200 | <200 |
| | | pre | <200 | <200 | <200 | <200 |
| | | just after/before 1^{st} | <200 | <200 | <200 | <200 |
| | | 2 weeks after 3^{rd} | 6913 | 5566 | 1081 | 16111 |
| | | 2 weeks after boost (4^{th}) = | 6000 | 5825 | 3735 | 14371 |
| | | 2 weeks before challenge | | | | |

Historical animals

| | | | | |
|---|---|---|---|---|
| Yoran | E1 mammalian | 1 week after last (6^{th}) = | 29836 | 32958 |
| | | 2 weeks before challenge | | |
| | | 2 weeks before challenge | | |
| Marti | E1 mammalian | 1 week after last (6^{th}) = | 13318 | 14280 |
| | | 2 weeks before challenge | | |
| Huub | Placebo | 1 week after last (6^{th}) = | <200 | <200 |
| | | 2 weeks before challenge | | |

### REFERENCES

1. Bedossa, P. and Poynard, T. (1996) Hepatology 24, 289-293.

2. Burns, J., Butler, J., and Whitesides, G. (1991) J. Org. Chem. 56, 2648-2650.

3. Choo, Q. L., Kuo, G., Ralston, R., Weiner, A., Chien, D., Van Nest, G., Han, J., Berger, K., Thudium, K., Kuo, C., and. (1994) Proc. Natl. Acad. Sci. U.S.A. 91, 1294-1298.

4. Cooper, S., Erickson, A. L., Adams, E. J., Kansopon, J., Weiner, A. J., Chien, D. Y., Houghton, M., Parham, P., and Walker, C. M. (1999) Immunity. 10, 439-449.

5. Darbre, A. (1986) Practical protein chemistry: a handbook Whiley & Sons Ltd.

6. Diepolder, H. M., Zachoval, R., Hoffmann, R. M., Wierenga, E. A., Santantonio, T., Jung, M. C., Eichenlaub, D., and Pape, G. R. (1995) Lancet 346, 1006-1007.

7. Diepolder, H. M., Gerlach, J. T., Zachoval, R., Hoffmann, R. M., Jung, M. C., Wierenga, E. A., Scholz, S., Santantonio, T., Houghton, M., Southwood, S., Sette, A., and Pape, G. R. (1997) J. Virol. 71, 6011-6019.

8. Erickson, A. L., Kimura, Y., Igarashi, S., Eichelberger, J., Houghton, M., Sidney, J., McKinney, D., Sette, A., Hughes, A. L., and Walker, C. M. (2001) Immunity. 15, 883-895.

9. Falck-Ytter, Y., Kale, H., Mullen, K. D., Sarbah, S. A., Sorescu, L., and McCullough, A. J. (2002) Ann. Intern. Med. 136, 288-292.

10. Forms, X., Emerson, S. U., Tobin, G. J., Mushahwar, I. K., Purcell, R. H., and Bukh, J. (1999) Vaccine 17, 1992-2002.

11. Forns, X., Payette, P. J., Ma, X., Satterfield, W., Eder, G., Mushahwar, I. K., Govindarajan, S., Davis, H. L., Emerson, S. U., Purcell, R. H., and Bukh, J. (2000) Hepatology 32, 618-625.

12. Frankel, A. E., McCubrey, J. A., Miller, M. S., Delatte, S., Ramage, J., Kiser, M., Kucera, G. L., Alexander, R. L., Beran, M., Tagge, E. P., Kreitman, R. J., and Hogge, D. E. (2000) Leukemia 14, 576-585.

13. Fujihashi, K., McGhee, J. R., Beagley, K. W., McPherson, D. T., McPherson, S. A., Huang, C. M., and Kiyono, H. (1993) J. Immunol. Methods 160, 181-189.

14. Gailit, J. (1993) Anal. Biochem. 214, 334-335.

15. Gosert, R. and Moradpour, D. (2002) Hepatology 36, 757-760.

16. Grakoui, A., Wychowski, C., Lin, C., Feinstone, S. M., and Rice, C. M. (1993) J. Virol. 67, 1385-1395.

17. Hermanson, G. T. (1996) Bioconjugate techniques Academic Press, San Diego.

18. Holmgren, A. (1979) J. Biol. Chem. 254, 9627-9632.

19. Houghton, M., Choo, Q. L., Kuo, G., Weiner, A., Chien, D., Ralston, R., Urdea, M., Moss, B., Purcell, R., Cummins, L., and. (1995) Princess Takamatsu Symp. 25, 237-243.

20. Hu, G. J., Wang, R. Y., Han, D. S., Alter, H. J., and Shih, J. W. (1999) Vaccine 17, 3160-3170.

21. Ishak, K., Baptista, A., Bianchi, L., Callea, F., De Groote, J., Gudat, F., Denk, H., Desmet, V., Korb, G., MacSween, R. N., and. (1995) J. Hepatol. 22, 696-699.

22. Ishii, K., Rosa, D., Watanabe, Y., Katayama, T., Harada, H., Wyatt, C., Kiyosawa, K., Aizaki, H., Matsuura, Y., Houghton, M., Abrignani, S., and Miyamura, T. (1998) Hepatology 28, 1117-1120.

23. Johnson, D. A., Sowell, C. G., Johnson, C. L., Livesay, M. T., Keegan, D. S., Rhodes, M. J., Ulrich, J. T., Ward, J. R., Cantrell, J. L., and Brookshire, V. G. (1999) Bioorg. Med. Chem Lett 9, 2273-2278.

24. Knodell, R. G., Ishak, K. G., Black, W. C., Chen, T. S., Craig, R., Kaplowitz, N., Kiernan, T. W., and Wollman, J. (1981) Hepatology 1, 431-435.

25. Koziel, M. J. and Liang, T. J. (1997) Gastroenterology 112, 1410-1414.

26. Kumar, N., Kella, D., and Kinsella, J. E. (1985) J. Biochem. Biophys. Methods 11, 251-263.

27. Kumar, N., Kella, D., and Kinsella, J. E. (1986) Int. J. Peptide Prot. Res. 28, 586-592.

28. Lauer, G. M. and Walker, B. D. (2001) N. Engl J. Med. 345, 41-52.

29. Lopez-Dias de Cerio A L, Casares, N., Lasarte, J. J., Sarobe, P., Perez-Mediavilla, L. A., Ruiz, M., Prieto, J., and Borras-Cuesta, F. (1999) Int Immunol. 11, 2025-2034.

30. Manns, M. P., McHutchison, J. G., Gordon, S. C., Rustgi, V. K., Shiffman, M., Reindollar, R., Goodman, Z. D., Koury, K., Ling, M., and Albrecht, J. K. (2001) Lancet 358, 958-965.

31. Missale, G., Bertoni, R., Lamonaca, V., Valli, A., Massari, M., Mori, C., Rumi, M. G., Houghton, M., Fiaccadori, F., and Ferrari, C. (1996) J. Clin. Invest 98, 706-714.

32. Park, J. H., Kwon, H. W., Chung, H. K., Kim, I. H., Ahn, K., Choi, E. J., Pastan, I., and Choe, M. (2001) Mol Cells 12, 398-402.

33. Persing, D., Coler, R., Lacy, M., Johnson, D., Baldridge, J., Hershberg, R., and Reed, S. (2002) Trends Microbiol. 10, S32.

34. Poynard, T., Bedossa, P., and Opolon, P. (1997) Lancet 349, 825-832.

35. Rein, A., Ott, D. E., Mirro, J., Arthur, L. O., Rice, W., and Henderson, L. E. (1996) J. Virol. 70, 4966-4972.

36. Riedl, P., Buschle, M., Reimann, J., and Schirmbeck, R. (2002) Eur. J. Immunol. 32, 1709-1716.

37. Shiffman, M. L. (1999) Viral Hepatitis Reviews 5, 27-43.

38. Shimizu, Y. K., Hijikata, M., Iwamoto, A., Alter, H. J., Purcell, R. H., and Yoshikura, H. (1994) J. Virol. 68, 1494-1500.

39. Shimotohno, K., Tanji, Y., Hirowatari, Y., Komoda, Y., Kato, N., and Hijikata, M. (1995) J. Hepatol. 22, 87-92.

40. Shirai, M., Chen, M., Arichi, T., Masaki, T., Nishioka, M., Newman, M., Nakazawa, T., Feinstone, S. M., and Berzofsky, J. A. (1996) J. Infect. Dis. 173, 24-31.

41. Singh, R. and Kats, L. (1995) Anal. Biochem. 232, 86-91.

42. Song, M. K., Lee, S. W., Suh, Y. S., Lee, K. J., and Sung, Y. C. (2000) J. Virol. 74, 2920-2925.

43. Thakur, M. L., DeFulvio, J., Richard, M. D., and Park, C. H. (1991) Int. J. Rad. Appl. Instrum. B 18, 227-233.

44. Xu, Z., Choi, J., Yen, T. S., Lu, W., Strohecker, A., Govindarajan, S., Chien, D., Selby, M. J., and Ou, J. (2001) EMBO J. 20, 3840-3848.

1. An HCV immunogenic composition comprising at least one HCV envelope peptide, at least one HCV non-structural peptide, and, optionally, a pharmaceutically acceptable carrier.

2. A HCV vaccine composition comprising an effective amount of at least one HCV envelope peptide and at least one HCV non-structural peptide, and, optionally, a pharmaceutically acceptable carrier.

3. A HCV vaccine composition according to claim 2, wherein said composition is a prophylactic HCV vaccine composition.

4. A HCV vaccine composition according to claim 2, wherein said composition is a therapeutic HCV vaccine composition.

5. The composition according to any of claims 1 to 4 wherein said HCV envelope peptide is an E1 peptide and wherein said HCV non-structural peptide is an NS3 peptide.

6. The composition according to claim 5 wherein said HCV E1 peptide is consisting of the HCV polyprotein region spanning amino acids 192 to 326.

7. The composition according to claim 5, wherein said E1 peptide is produced by expression in yeast.

8. The composition according to claim 7 wherein said yeast is Hansenula polymorpha.

9. The composition according to claim 5 wherein said HCV NS3 peptide is comprising the HCV polyprotein region spanning amino acids 1188 to 1468 and/or HCV polyprotein region spanning amino acids 1071 to 1084 or parts thereof.

10. The composition according to claim 5 wherein said HCV E1 peptide is defined by SEQ ID NO:1.

11. The composition according to claim 9 wherein said HCV polyprotein region spanning amino acids 1188 to 1468 is defined by SEQ ID NO:2.

12. The composition according to claim 9 wherein said HCV polyprotein region spanning amino acids 1071 to 1084 is defined by SEQ ID NO:3.

13. The composition according to claim 9 wherein said part of said HCV polyprotein region spanning amino acids 1071 to 1084 is the HCV polyprotein region spanning amino acids 1073 to 1081.

14. The composition according to claim 13 wherein said part of said HCV polyprotein region spanning amino acids 1073 to 1081 is defined by SEQ ID NO:4.

15. The composition according to claim 5 wherein said HCV NS3 peptide is defined by SEQ ID NO:5.

16. The composition according to any of claims 1 to 4 wherein said HCV peptides are linked, optionally via a spacer.

17. The composition according to any of claim 1 to 4 wherein said HCV peptides are synthetic peptides or recombinant peptides.

18. The composition according to any of claims 1 to 4 wherein at least one cysteine of said HCV peptides are reversibly or irreversibly blocked.

19. The composition according to any of claims 1 to 4 wherein at least one cysteine of said HCV envelope peptide is alkylated.

20. The composition according to any of claims 1 to 4 wherein at least one cysteine of said HCV non-structural peptide is sulphonated.

21. The composition according to any of claims 1 to 4 wherein said HCV envelope peptide is added to said composition as viral-like particles.

22. The composition according to any of claims 1 to 4 wherein said pharmaceutically acceptable carrier is alum.

23. The composition according to any of claims 1 to 4 comprising a plurality of HCV envelope peptides derived from different HCV genotypes, subtypes or isolates and/or a plurality of HCV non-structural peptides derived from different HCV genotypes, subtypes or isolates.

24. A method for inducing a humoral response to the HCV peptides comprised in a composition according to any of claims 1 to 4, said method comprising administering said composition to a mammal.

25. A method for inducing a cellular response to the HCV peptides comprised in a composition according to any of claims 1 to 4, said method comprising administering said composition to a mammal.

26. The method according to claim 25 wherein said cellular response is a CD4+ T-cell proliferation response and/or a CD8+ cytotoxic T-cell response and/or a cytokine secretion response.

27. A method for prophylactic protection of a mammal against chronic HCV infection, said method comprising administering a composition according to any of claims 1 to 4 to said mammal.

28. A method for prophylactic protection of a mammal against chronic infection by a homologous or heterologous HCV, said method comprising administering a composition according to any of claims 1 to 4 to said mammal.

29. A method for therapeutically treating a chronically HCV-infected mammal, said method comprising administering a composition according to any of claims 1 to 4 to said mammal.

30. A method for therapeutically treating a mammal chronically infected with a homologous or heterologous HCV, said method comprising administering a composition according to any of claims 1 to 4 to said mammal.

31. A method for reducing liver disease in a HCV-infected mammal, said method comprising administering a composition according to any of claims 1 to 4 to said mammal.

32. A method for reducing liver disease in a HCV-infected mammal by at least 2 points according to the overall Ishak score comprising administering a composition according to any of claims 1 to 4 to said mammal.

33. A method for reducing serum liver enzyme activity levels in a HCV-infected mammal, said method comprising administering a composition according to any of claims 1 to 4 to said mammal.

34. A method for reducing HCV RNA levels in a HCV-infected mammal, said method comprising administering a composition according to any of claims 1 to 4 to said mammal.

35. A method for reducing liver fibrosis progression in a HCV-infected mammal, said method comprising administering a composition according to any of claims 1 to 4 to said mammal.

36. A method for reducing liver fibrosis in a HCV-infected mammal, said method comprising administering a composition according to any of claims 1 to 4 to said mammal.

37. A method for vaccinating a HCV-naïve or HCV-infected mammal comprising administering a DNA vaccine and a composition according to any of claims 1 to 4.
